SEQUENCE LISTING (36 sequences)

SEQ ID NO: 1 - DNA, 1161 bp, Bacillus anthracis
  ref <308>: DATABASE ACCESSION NUMBER: GenBank Acc. # AE017030 (locus tag BA1995)
  ref <309>: DATABASE ENTRY DATE: 2003-04-30
  ref <313>: RELEVANT RESIDUES: (1)..(1161)
atgggaaagacaagtaagtatgtgacagctgccgcactttgttcaactatagtaatggggggcttacacgcgtcatctgtatcttatgcagctacgaatccaactgtagcgacattacaatcagatgcaaagttgttaaatgatttcaaaaaagaattaaaaaaacagattgataatcgagaagaaaatattacaatcacatataaaacaaaagatagaaatgctagaaatattatggatgaattgtatggcgagtttaataaaattgtagatgcagatgagtatgtaaaatataatatagcatctactagatattccattaaagggttaccaggaaactatacgtttacactgcaggtgaagtaccgtgaatcaaaagagcaaacacaatatgtaaaatctcaggcaaaagcaattatcggctcgattgtaaaaccaggaatggatgagcatgagaaagtaaaagctattcatgattacgttgtaaaacatgtatcgtatgacacgtcgtatcaagcatatacagcatatgaagcgttagcgaatcgctctgctgtgtgccaaggatatacgttattaacatatgaattactaaaagaagcgggtatccaaaaccgcattgtaacaggcacaggaaatggacaagctcactcatggaatttagtaaacattgaaaacaaatggtatcatcttgatacgacattcgatgatccagtaccagataaagctgggcgcgtaacatattcatattttaatatgtctgacgagcaattaagtaaagatcacgactgggatcgtagtaaatatccagcagcaactacaagctactttaatgaattaacaaacaaaataaaagctggtagttcaaaaactgctacatatgaacaaatgttaaaagaaacaaatttaaaatatttgtctgcacaatacggggcagacaattacagtgaatttaaggaaaaattacagcaacaattcgcttctaagccagagaaggtagaagtacgctataagcaatccatggatggaacaatgcaagatataaagaaagtgttaaatgaaataaactggccaaaaggtgcaaagcgtgtatcttatcaagtagcaccatatagtgctatggcagattattcattagcgacaattacatttacgtattaa

SEQ ID NO: 2 - DNA, 1161 bp, Bacillus anthracis; identical to SEQ ID NO: 1
  CDS (1)..(1161)

SEQ ID NO: 3 - protein, 386 aa, Bacillus anthracis
MGKTSKYVTAAALCSTIVMGGLHASSVSYAATNPTVATLQSDAKLLNDFKKELKKQIDNREENITITYKTKDRNARNIMDELYGEFNKIVDADEYVKYNIASTRYSIKGLPGNYTFTLQVKYRESKEQTQYVKSQAKAIIGSIVKPGMDEHEKVKAIHDYVVKHVSYDTSYQAYTAYEALANRSAVCQGYTLLTYELLKEAGIQNRIVTGTGNGQAHSWNLVNIENKWYHLDTTFDDPVPDKAGRVTYSYFNMSDEQLSKDHDWDRSKYPAATTSYFNELTNKIKAGSSKTATYEQMLKETNLKYLSAQYGADNYSEFKEKLQQQFASKPEKVEVRYKQSMDGTMQDIKKVLNEINWPKGAKRVSYQVAPYSAMADYSLATITFTY

SEQ ID NO: 4 - DNA, 1161 bp, Bacillus cereus
  ref <308>: DATABASE ACCESSION NUMBER: GenBank Acc. # AE017004 (locus tag BC1991)
  ref <309>: DATABASE ENTRY DATE: 2003-05-16
  ref <313>: RELEVANT RESIDUES: (1)..(1161)
atgggaaagacaagtaagtatgtgacagctgccgcactttgttcaactatagtaatgggaggcttacaagcatcatctgtatcttatgcagctacaaatccgactacagtgacagcacaatcagatgtaaagttattagatgatttcagaaaagaattaaaaaaacagattgataatcgagaagaaaatattacaatcacatataaaacaaaagatagaaatgctagaaatattatggatcaattgtatggtgagtttaataaaattgtagatgctgatgagtatgtaaaatataatgtagcgtctactagatattctattaaaggaatgccaggggactatacgtttacactgcaagtaaaataccgtgaatcaaaagaacaaactcaatatgtaaaagctcaggcgaaatcaattgttggatcaattgtaaaaccagggatggatgagcatgagaaagtaaaagctattcacgattacgttgttaagcatgtgtcttatgatacgtcttatcaagcgtatacagcatatgaagcattagcgaatcgttctgccgtttgccaaggatatacattattaacgtatgagttgttaaaagaggcaggtattcaaaatcatattgtaacaggcacaggaaatggacaagctcacgcgtggaatttagtgaacattgaaaacaaatggtatcacctagatactacatttgatgatccagtaccagataaagcagggcgtgtaacttactcatattttaatatgtctgacgaacagttaagtaaagatcatgaatgggatcgtagcaaatatccagcggcaactacaagttattttggtgaattaacaaataaaataaaagctggtagctcaaagactgtcgtatatgagcagatgttaaaagaaacaaatttacaatacttatctgcagaatacggagcagaaaattataatgaatttaagcaaaagttgcagcaacagtttgcagccaaaccagaaaaagtagaagtacgatataagcagtccatggatgggacgatgcaagatataaagaaagtattaaatgaaataaattggccaaaaggtgcaaagcgtgtgtcttatcaagtagcgccttatagtgcattggctggttattcattggcgacaattacatttacgtattaa

SEQ ID NO: 5 - DNA, 1161 bp, Bacillus cereus; identical to SEQ ID NO: 4
  CDS (1)..(1161)

SEQ ID NO: 6 - protein, 386 aa, Bacillus cereus
MGKTSKYVTAAALCSTIVMGGLQASSVSYAATNPTTVTAQSDVKLLDDFRKELKKQIDNREENITITYKTKDRNARNIMDQLYGEFNKIVDADEYVKYNVASTRYSIKGMPGDYTFTLQVKYRESKEQTQYVKAQAKSIVGSIVKPGMDEHEKVKAIHDYVVKHVSYDTSYQAYTAYEALANRSAVCQGYTLLTYELLKEAGIQNHIVTGTGNGQAHAWNLVNIENKWYHLDTTFDDPVPDKAGRVTYSYFNMSDEQLSKDHEWDRSKYPAATTSYFGELTNKIKAGSSKTVVYEQMLKETNLQYLSAEYGAENYNEFKQKLQQQFAAKPEKVEVRYKQSMDGTMQDIKKVLNEINWPKGAKRVSYQVAPYSALAGYSLATITFTY

SEQ ID NO: 7 - DNA, 2331 bp, Bacillus anthracis
  ref <308>: DATABASE ACCESSION NUMBER: GenBank Acc. # AE017034 (locus tag BA3313)
  ref <309>: DATABASE ENTRY DATE: 2003-04-30
  ref <313>: RELEVANT RESIDUES: (1)..(2331)
atgaaaaaagtaataaatatgtttataaaaaagtatcaagttaaaggcattgtgcaaggtgttggttttagacctttcgtatataggatagcaaaaaagtatcatctaaacggatgggtgttaaatgattcaaatggagtgcttatagaagtagagggggaacaaaataatatttcgcattttattcatgatattgaatttaattccccagaattggcttatgtgcaagaggttaaattgatagaggaacaagaatccaacaatcgttatagctcttttgatattttgagtagccaaaaacatagtagtaggcaaaccttaatatctcctgatacttatgtatgtaatgaatgtttagaagaattatttgatccaacgaatagaaggtttcgttatccatttattaattgtacaaattgtggacctagatattcaataataaaagatattccttatgatagaccgtatactacaatgtccaaatttcctatgtgcaaagagtgtgatatagaatacaaagacatacttgatcggagatttcatgcacaaccaaatgcttgctggaaatgtggaccacaagttcaattgaaaacaacagatggtacaaatgtctccactgataatccaatagaaaaaacaattgatttgttgaaaaaggggaaaatcctagctattaaagggttaggtggatatcacttagttgtagatccaatgaatcatgaagctgtaaaagaattaagaaaaagaaaaaaacgggatgaaaaaccttttgctttaatgtcatctcaattagatgatgtgaaaaattttgctgaaataacaaaaaatgaagaaactttattagtttctaaagaacgaccaattgtgttattgaacaaaaaggataatgagtatatttcttcagatgttgcatctagtaataatacctatggggtaatgttagcctatacaccacttcatcatcttattttaagagggaattttatttcattaatagctacgagcgccaatgtttctgatgaacctattgtatataaagatggagatgcaattgagcagctaaagggaattgcagattattatttagtacacaatagggagatattcactagagtagatgactctattgttagggggattgatttagatgatgaagtaataaaactacaaataattcgtcgcgctagaggttatgtaccaaacccaattgattttaaagaaggatcacctaatgtacttgctttaggagcagaattaaagaatacgatttgcttaactaaaggtgaaaaagcttttattagtcatcatattggtgatttaaaaaactataaaatttatgaatcgtttaaaaatactatagctcacttacaaaaaatctttgaaattagtccttctgtaattgcaagtgatcttcatcctgatttttatagtacaaaatatgcatttgatcaaaatgttttaccagttgtacaggttcagcatcatcatgcccacatggcttcttgtatggttgataatcaattagatggtcctgttttaggagttattttagatggtacgggttatggtgatgatgggaatatttggggtggagagtttttaatcggtgattttcttaattatgaaagagcggcatatatcgattactttcagctgccaggcggtgataaagctgttaaagaaccgtatagaattgctttaggaatattgacgcaaatatatggtgaacaagtcaaagaattaccaataaagataatttcagagcgaaatagttttgaactagatgtacttataaaaatgattcaaaaaggaattaattctcctcttacttcaagtatgggaaggctttttgatgctgtttcagcactattgggtgtctgtgaaacagtccaatatgaaggacaggctgctattgagttggagcaagcgattgatatcaataattcttccaatcctttattatcatatgaaatagttgcttcgaatggaattagaaaaatcgatataaaacctatgtttaaagagttggtatctttgatacttaaagataagtataccaagggtgaattgagctatcgttttcattatacaatggttcaaatgattacagaaacctgtaaacaaattcgtcaagaaacagggattaataaactagttcttagtgggggagtatttttaaataagtttttacttgtagaaacctataagtcacttaaggcttgtaaattcgaggtttacatgcacaaacaagtacctaccaatgatgggggaatctctcttggtcaagcagcaatagcagctcatcgctataaagatatgcattcggtataa

SEQ ID NO: 8 - DNA, 2331 bp, Bacillus anthracis; identical to SEQ ID NO: 7
  CDS (1)..(2331)

SEQ ID NO: 9 - protein, 776 aa, Bacillus anthracis
MKKVINMFIKKYQVKGIVQGVGFRPFVYRIAKKYHLNGWVLNDSNGVLIEVEGEQNNISHFIHDIEFNSPELAYVQEVKLIEEQESNNRYSSFDILSSQKHSSRQTLISPDTYVCNECLEELFDPTNRRFRYPFINCTNCGPRYSIIKDIPYDRPYTTMSKFPMCKECDIEYKDILDRRFHAQPNACWKCGPQVQLKTTDGTNVSTDNPIEKTIDLLKKGKILAIKGLGGYHLVVDPMNHEAVKELRKRKKRDEKPFALMSSQLDDVKNFAEITKNEETLLVSKERPIVLLNKKDNEYISSDVASSNNTYGVMLAYTPLHHLILRGNFISLIATSANVSDEPIVYKDGDAIEQLKGIADYYLVHNREIFTRVDDSIVRGIDLDDEVIKLQIIRRARGYVPNPIDFKEGSPNVLALGAELKNTICLTKGEKAFISHHIGDLKNYKIYESFKNTIAHLQKIFEISPSVIASDLHPDFYSTKYAFDQNVLPVVQVQHHHAHMASCMVDNQLDGPVLGVILDGTGYGDDGNIWGGEFLIGDFLNYERAAYIDYFQLPGGDKAVKEPYRIALGILTQIYGEQVKELPIKIISERNSFELDVLIKMIQKGINSPLTSSMGRLFDAVSALLGVCETVQYEGQAAIELEQAIDINNSSNPLLSYEIVASNGIRKIDIKPMFKELVSLILKDKYTKGELSYRFHYTMVQMITETCKQIRQETGINKLVLSGGVFLNKFLLVETYKSLKACKFEVYMHKQVPTNDGGISLGQAAIAAHRYKDMHSV

SEQ ID NO: 10 - DNA, 2130 bp, Bacillus cereus
  ref <308>: DATABASE ACCESSION NUMBER: GenBank Acc. # AE017008 (locus tag BC3241)
  ref <309>: DATABASE ENTRY DATE: 2003-05-16
  ref <313>: RELEVANT RESIDUES: (1)..(2130)
atgaaaaaagtaataaatatgtttataaaaaagtatcaagttaaaggcattgtgcaaggtgttggttttagacctttcgtatataggatagcaaaaaagtatcatctaaacggatgggtgttaaatgattcaaatggagtgcttatagaagtagaaggggaacaaaataatattacgcattttattcatgatattgaatttaattccccagaattggcttatgtgcaagaggttaaattaatagaggaacaagaatccaacaatcgttatagctcttttgatattttgagtagccaaaaacatagtagtaggcaaactttaatatctcctgatacttatgtatgtaatgaatgtttagacgaattatttgatccaacgaatagaaggtttcgttatccatttattaattgtacaaattgtggacctagatattcaataataaaagatattccttatgatagaccatatactacaatggccaaatttcctatgtgcaaagagtgtgataaagaatacaaagacatattggatcggagattccatgcccaaccaaatgcttgctggaaatgtggaccacaagttcaattgaaaacaacagatggtacaaatgtctccactgataatccaatagaaaaaacaattgatttgttgaaaaaggggaaaatcctagctattaaagggttaggtggatatcacttagttgtagatccaatgaatcatgaagctgtaaaagaattaagaaaaagaaaaaaacgggatgaaaaaccttttgctttaatgtcatctcaattagatgatgtgaaaaattttgctgaaataacaaaaaatgaagaaactttattagtttctaaagaacgaccaattgtgttattgaacaaaaaggataatgagtttatttctttagatgttgcaccaaataataataattatggggtaatgttagcctatacaccacttcatcatcttattttaagagggaattttattgcattaatagctacgagcgccaatgtttctgatgaacctattgtatataaagatggagatgcaattgagcagctaaagggaattgcagattattatttagtacacaatagggagatattcactagagtagatgactctattgttaggggtattgatttagatgatgaagtaataaaactacaaataattcgtcgcgctagaggttatgtaccaaacccaattgattttaaagaaggatcaactaatgtacttgctttaggagcagaattaaagaatacgatttgcctaactaaagacgaaaaagcttttattagtcatcgtattggtgatttaaaaaactataaaatttatgaatcgtttaaaaatactatagctcacttacaaaaaatctttgaaattagtccttctgtaattgcaagtgatcttcatcctgatttttatagtacaaaatatgcatttgatcaaaaagttttaccagttgtacaggttcagcatcatcatgcccacatggcttcttgtatggttgataatcaattagatggtcctgttttaggagttattttagatggtacgggttatggtgatgatgggaatatttggggtggagagtttttaatcggtgattttcttaattatcaaagagcggcatatatcgattactttcagctgccaggcggtgataaagctgttaaagaaccgtatagaattgctttaggaatattgacgcaaatatatggtgaacaagtcaaagaattaccaataaagataatttcagagcgaaatagttttgaactagatgtacttataaaaatgattcaaaaaggaattaattctcctcttacttcaagtatgggaaggctttttgatgctgtttcagcactattgggtgtccgtgaaacagtccaatatgaaggacaggctgctattgagttggagcaagcgattgatatcaataattcttccaatcctttattatcatatgaaatagttgcttcgaatggaattagaaaaatcgatataaaacctatgtttaaagagttggtatctttgatacttaaagataagtataccaagggtgaattgagctatcgttttcattatacaatggtgcattcaagaaaatgtgaaaataatgaatattag

SEQ ID NO: 11 - DNA, 2130 bp, Bacillus cereus; identical to SEQ ID NO: 10
  CDS (1)..(2130)

SEQ ID NO: 12 - protein, 709 aa, Bacillus cereus
MKKVINMFIKKYQVKGIVQGVGFRPFVYRIAKKYHLNGWVLNDSNGVLIEVEGEQNNITHFIHDIEFNSPELAYVQEVKLIEEQESNNRYSSFDILSSQKHSSRQTLISPDTYVCNECLDELFDPTNRRFRYPFINCTNCGPRYSIIKDIPYDRPYTTMAKFPMCKECDKEYKDILDRRFHAQPNACWKCGPQVQLKTTDGTNVSTDNPIEKTIDLLKKGKILAIKGLGGYHLVVDPMNHEAVKELRKRKKRDEKPFALMSSQLDDVKNFAEITKNEETLLVSKERPIVLLNKKDNEFISLDVAPNNNNYGVMLAYTPLHHLILRGNFIALIATSANVSDEPIVYKDGDAIEQLKGIADYYLVHNREIFTRVDDSIVRGIDLDDEVIKLQIIRRARGYVPNPIDFKEGSTNVLALGAELKNTICLTKDEKAFISHRIGDLKNYKIYESFKNTIAHLQKIFEISPSVIASDLHPDFYSTKYAFDQKVLPVVQVQHHHAHMASCMVDNQLDGPVLGVILDGTGYGDDGNIWGGEFLIGDFLNYQRAAYIDYFQLPGGDKAVKEPYRIALGILTQIYGEQVKELPIKIISERNSFELDVLIKMIQKGINSPLTSSMGRLFDAVSALLGVRETVQYEGQAAIELEQAIDINNSSNPLLSYEIVASNGIRKIDIKPMFKELVSLILKDKYTKGELSYRFHYTMVHSRKCENNEY

SEQ ID NO: 13 - DNA, 354 bp, Bacillus anthracis
  ref <308>: DATABASE ACCESSION NUMBER: GenBank Acc. # AE017035 (locus tag BA3509)
  ref <309>: DATABASE ENTRY DATE: 2003-05-01
  ref <313>: RELEVANT RESIDUES: (1)..(354)
atgataacacctatatttagaatttttgatattgaaaaagcaaagctattttacgtaggttttttaggatttaaactggattgggaacatcgttatgaggaacatatgccattatatatgcaaatttcgttacatgatgctgtgatgcatttatcagaacatcatggtgatgcctcaccaggtggtgcgattcggatcaaaatagatgatgtaaaggattatcattctgtattattaagtaaagaatatgcttattcgaagccgaatatagaaaaaacaccttggggtacaattgaattaactgtgattgatccgttttcaaatagaattatattttatgaggagagggtttag

SEQ ID NO: 14 - DNA, 354 bp, Bacillus anthracis; identical to SEQ ID NO: 13
  CDS (1)..(354)

SEQ ID NO: 15 - protein, 117 aa, Bacillus anthracis
MITPIFRIFDIEKAKLFYVGFLGFKLDWEHRYEEHMPLYMQISLHDAVMHLSEHHGDASPGGAIRIKIDDVKDYHSVLLSKEYAYSKPNIEKTPWGTIELTVIDPFSNRIIFYEERV

SEQ ID NO: 16 - DNA, 354 bp, Bacillus cereus
  ref <308>: DATABASE ACCESSION NUMBER: GenBank Acc. # AE017009 (locus tag BC3448)
  ref <309>: DATABASE ENTRY DATE: 2003-05-16
  ref <313>: RELEVANT RESIDUES: (1)..(354)
atgataacacctatatttagaatttttgatattgaaaaagcaaaactattttacttaggttttttaggatttaaactggattgggaacatcggtatgaggaaaatatgccattatatttgcaaatttcgttacaggatgctgtgatacatttatcagaacatcatggtgatgcctcaccaggtggtgcgattcggatcaaaatagatgatgtaaaggattatcattctgtattattaagtaaagaatatgcttattcgaagccgaatatagaaaaaacaccttggggtacaattgaattaactgtaattgatccgttttcaaatagaattacactatacgaggaaaaattgtag

SEQ ID NO: 17 - DNA, 354 bp, Bacillus cereus; identical to SEQ ID NO: 16
  CDS (1)..(354)

SEQ ID NO: 18 - protein, 117 aa, Bacillus cereus
MITPIFRIFDIEKAKLFYLGFLGFKLDWEHRYEENMPLYLQISLQDAVIHLSEHHGDASPGGAIRIKIDDVKDYHSVLLSKEYAYSKPNIEKTPWGTIELTVIDPFSNRITLYEEKL

SEQ ID NO: 19 - DNA, 615 bp, Bacillus anthracis
  ref <308>: DATABASE ACCESSION NUMBER: GenBank Acc. # AE017028 (locus tag BA1500)
  ref <309>: DATABASE ENTRY DATE: 2003-04-30
  ref <313>: RELEVANT RESIDUES: (1)..(615)
atgaaagttttaaaatgcggtgtcatgttattgagtatactgtttgtatctcaattacatgtttatgcagaagaaaatcgatggacatggcctgttgaaggtcagataagtgattattttgggacaaggcatggaaaacactatggtattgatgtagctgcgccgattggaacgcctgtggtagctatccaagatggtaaggtaacgaggtcttattattcaagtagttatggaaatgttgtatttattaaacacggagaatatgaggctgtatatgcacatttaaataagagatatgtgaatcaaggagattatatttccaaaggagaaaaaattggagaagtagggaacacgggagaatcgcgaggtgcgcacttacatctagaacttcatcaaggaagatggacgatggcgaaaaagaatgcgatgaacccattgcttgttttaagtgaacaaagaaatgaagttgtttcttcgtcattatatgtcgtacaaaaaggagatactttagttagtattgcacggaaatttagtatgacacttaaggaaattaaagaaaaaaatggattgcagcaagagctaatttatcctaatcaacaattatatgttaagtaa

SEQ ID NO: 20 - DNA, 615 bp, Bacillus anthracis; identical to SEQ ID NO: 19
  CDS (1)..(615)

SEQ ID NO: 21 - protein, 204 aa, Bacillus anthracis
MKVLKCGVMLLSILFVSQLHVYAEENRWTWPVEGQISDYFGTRHGKHYGIDVAAPIGTPVVAIQDGKVTRSYYSSSYGNVVFIKHGEYEAVYAHLNKRYVNQGDYISKGEKIGEVGNTGESRGAHLHLELHQGRWTMAKKNAMNPLLVLSEQRNEVVSSSLYVVQKGDTLVSIARKFSMTLKEIKEKNGLQQELIYPNQQLYVK

SEQ ID NO: 22 - DNA, 615 bp, Bacillus cereus
  ref <308>: DATABASE ACCESSION NUMBER: GenBank Acc. # AE017002 (locus tag BC1480)
  ref <309>: DATABASE ENTRY DATE: 2003-05-16
  ref <313>: RELEVANT RESIDUES: (1)..(615)
atgaaagttttaaaatgcggtgtcatgttattgagtatactgtttgtatctcaattacatgtttatgcagaagaaaatcgatggacatggcctgttgaaggtcagataagtgattattttgggacaaggcatggaaaacactatggtattgatgtagctgcgccgattggaacgcctgtggcagctatccaagatggtaaggtaacgaggtcttattattcaagtagttatggaaatgttgtatttattaaacacggagaatatgaggctgtatatgcacatttaaataagagatatgtggatcaaggagattatatttccaaaggagaaaaaattggagaagtagggaacacgggagaatcgcgaggtgcgcacttacatctagaacttcatcaaggaagatggacgatggcgaaaaagaatgcgatgaacccattgcttgttttaagtgaacaaagaaatgaagttgtttcttcgtcattatatgtcgtacaaaaaggggatactttagttagtattgcacggaaatttattatgacacttaaggaaattaaagaaaaaaatggattgcagcaagagctaatttatcctaatcaacaattatatgttaagtaa

SEQ ID NO: 23 - DNA, 615 bp, Bacillus cereus; identical to SEQ ID NO: 22
  CDS (1)..(615)

SEQ ID NO: 24 - protein, 204 aa, Bacillus cereus
MKVLKCGVMLLSILFVSQLHVYAEENRWTWPVEGQISDYFGTRHGKHYGIDVAAPIGTPVAAIQDGKVTRSYYSSSYGNVVFIKHGEYEAVYAHLNKRYVDQGDYISKGEKIGEVGNTGESRGAHLHLELHQGRWTMAKKNAMNPLLVLSEQRNEVVSSSLYVVQKGDTLVSIARKFIMTLKEIKEKNGLQQELIYPNQQLYVK

SEQ ID NO: 25 - DNA, 255 bp, Bacillus anthracis
  ref <308>: DATABASE ACCESSION NUMBER: GenBank Acc. # AE017039 (locus tag BA4865)
  ref <309>: DATABASE ENTRY DATE: 2003-04-30
  ref <313>: RELEVANT RESIDUES: (1)..(255)
gtgaataatcagaaaatttatgttatactatatgaaaaggaggttcatatgaggcaaatacatggagcaatttatatttacataacaatgtttttcgtggcgatatcttatggattagggcacgtgtattcgcatcctatactaacttttttaagcggagcatgtatggcgtttgcgcttcttgtccaccttttctcagtttggatcgtgaaatttcaattaaatattagtgaaatagaagaaggcactttctaa

SEQ ID NO: 26 - DNA, 255 bp, Bacillus anthracis; identical to SEQ ID NO: 25
  CDS (1)..(255)

SEQ ID NO: 27 - protein, 84 aa, Bacillus anthracis
VNNQKIYVILYEKEVHMRQIHGAIYIYITMFFVAISYGLGHVYSHPILTFLSGACMAFALLVHLFSVWIVKFQLNISEIEEGTF

SEQ ID NO: 28 - DNA, 255 bp, Bacillus cereus
  ref <308>: DATABASE ACCESSION NUMBER: GenBank Acc. # AE017013 (locus tag BC4618)
  ref <309>: DATABASE ENTRY DATE: 2003-05-16
  ref <313>: RELEVANT RESIDUES: (1)..(255)
gtgaataatcagaaaatttatgttataatatgggaaaaggaggtttatatgaggcaaatacatggagcaatttatatttacataacaatgtttttcgtggcggtatcctatggattggggcacgtgtattcgcatcctatactaacttttttaagcggagcatgtatggcgtttgcgcttcttgtccaccttttctcagtttggatcgtgaaatttcaattaaatattagtgaaatagaagaaggcactttttga

SEQ ID NO: 29 - DNA, 255 bp, Bacillus cereus; identical to SEQ ID NO: 28
  CDS (1)..(255)

SEQ ID NO: 30 - protein, 84 aa, Bacillus cereus
VNNQKIYVIIWEKEVYMRQIHGAIYIYITMFFVAVSYGLGHVYSHPILTFLSGACMAFALLVHLFSVWIVKFQLNISEIEEGTF

SEQ ID NO: 31 - DNA, 273 bp, Bacillus anthracis
  ref <308>: DATABASE ACCESSION NUMBER: GenBank Acc. # AE017029 (locus tag BA1598)
  ref <309>: DATABASE ENTRY DATE: 2003-04-30
  ref <313>: RELEVANT RESIDUES: (1)..(273)
atggaagtacataaagcaattacagcacattctcgtaaacaaaatgaaagtgtaaaagcatgtttacaattagatgcgcagcgtgaagcagctattgaagctgccgtatcacttgcgtcaaatgggaaagaattttcggttgatgtcattaatgttgtaacgaagcaaattaatgctcttgcaaaaaatggtgttaccttgcagcgtaaatatgttacaaaagaaatggttatggagtatgtaagtcgtttgaaagagaaagaaggtcgttaa

SEQ ID NO: 32 - DNA, 273 bp, Bacillus anthracis; identical to SEQ ID NO: 31
  CDS (1)..(273)

SEQ ID NO: 33 - protein, 90 aa, Bacillus anthracis
MEVHKAITAHSRKQNESVKACLQLDAQREAAIEAAVSLASNGKEFSVDVINVVTKQINALAKNGVTLQRKYVTKEMVMEYVSRLKEKEGR

SEQ ID NO: 34 - DNA, 273 bp, Bacillus cereus
  ref <308>: DATABASE ACCESSION NUMBER: GenBank Acc. # AE017003 (locus tag BC1575)
  ref <309>: DATABASE ENTRY DATE: 2003-05-16
  ref <313>: RELEVANT RESIDUES: (1)..(273)
atggaagtacataaagcaattacagcacattctcgtaaacaaaatgaaagtgtaaaagcatgtttacaattagatgcacagcgtgaagcagctattgaagctgccgtatctcttgcgtcaaatgggaaagaattttcggttgatgtcattaatgttgtaacgaagcaaattaatgatcttgcaaaaaatggtgttaccttgcagcgtaaatatgttacagaagaaatggttatggagtatgtaagtcgtttgaaaaagaaagaaggtcgttaa

SEQ ID NO: 35 - DNA, 273 bp, Bacillus cereus; identical to SEQ ID NO: 34
  CDS (1)..(273)

SEQ ID NO: 36 - protein, 90 aa, Bacillus cereus
MEVHKAITAHSRKQNESVKACLQLDAQREAAIEAAVSLASNGKEFSVDVINVVTKQINDLAKNGVTLQRKYVTEEMVMEYVSRLKKKEGR

(19) United States
(12) Patent Application Publication
(10) Pub. No.: US 2005/0181387 A1
(43) Pub. Date: Aug. 18, 2005

(54) Methods to identify evolutionarily significant changes in polynucleotide and polypeptide sequences in prokaryotes

(75) Inventor: Walter Messier, Longmont, CO (US)

Correspondence Address: SWANSON & BRATSCHUN L.L.C., 1745 SHEA CENTER DRIVE, SUITE 330, HIGHLANDS RANCH, CO, 80129, US

(73) Assignee: EVOLUTIONARY GENOMICS LLC

(21) Appl. No.: 10/956,353

(22) Filed: Oct. 1, 2004

Related U.S. Application Data
(60) Provisional application No. 60/507,988, filed on Oct. 1, 2003.

(51) Int. Cl. C12Q 1/68; C07H 21/04

(52) U.S. Cl. 435/6; 536/23.7

(57) ABSTRACT

Methods for identifying polynucleotide and polypeptide sequences which may be associated with commercially relevant or useful traits in prokaryotes are provided. The methods employ comparison of homologous genes from two closely related prokaryote species to identify evolutionarily significant changes. Sequences thus identified may be useful in developing therapeutics, diagnostics, or vaccines.

### CROSS-REFERENCE TO RELATED APPLICATIONS

This application claims the benefit, under 35 U.S.C. § 119 of U.S. Patent Application Ser. No. 60/507,988, file Oct. 1, 2003, entitled, “Methods to Identify Evolutionarily Significant Changes in Polynucleotide and Polypeptide Sequences in Prokaryotes,” which is incorporated by reference herein in its entirety.

### FIELD OF THE INVENTION

This invention relates to using molecular and evolutionary techniques to identify polynucleotide and polypeptide sequences corresponding to commercially relevant traits in prokaryotes.

### BACKGROUND OF THE INVENTION

The Centers for Disease Control classifies Bacillus anthracis among the agents considered the highest threat to national security because they are highly lethal and easily transmitted. Bioterrorism attacks in October 2001 using mailed B. anthracis spores resulted in 15 anthrax cases, 3 deaths, as well as public panic and disruption of government and the postal service.

Recent genomic analyses of the genomes of several Bacillus species indicates a very close relationship, with a strong likelihood that B. anthracis has undergone a selective (adaptive) shift towards greater virulence. Such selective shifts leave a diagnostic signature upon the genes that are responsible for altered functional traits associated with the selective shift. Thus, identification of positively selected genes in comparisons between B. anthracis, B. cereus, and B. thuringiensis should yield the chromosomal genes that code for virulence in B. anthracis.

By far, most genes are well suited to their functions and will not tolerate any changes. Adapted genes thus stand out from the norm because they have incorporated a statistically significant number of changes. Ka/Ks analysis (Li, et al. 1985. A new method for estimating synonymous and nonsynonymous rates of nucleotide substitution considering the relative likelihood of nucleotide and codon changes. Mol. Biol. Evol. 2: 150-174; Hughes, et al., 1988. Pattern of nucleotide substitution at major histocompatibility complex class I loci reveals overdominant selection. Nature 335: 167-170; Li 1993. Unbiased estimation of the rates of synonymous and nonsynonymous substitution. J. Mol. Evol. 36: 96-99; Li 1997. Molecular Evolution. Sinauer, Sunderland, Mass.; Messier & Stewart. 1997. Episodic adaptive evolution of primate lysozymes. Nature 385: 151-154) involves pairwise comparisons of homologous protein-coding genes of closely related species and calculation of the ratios of nonsynonymous nucleotide substitutions per nonsynonymous site (Ka) to synonymous substitutions per synonymous site (Ks) (where nonsynonymous means substitutions that change the encoded amino acid and synonymous means substitutions that do not change the encoded amino acid). Genes that have been subjected to positive selection display Ka/Ks ratios that are significantly greater than one. Genes that have been subjected to negative selection (strongly conserved) have Ka/Ks ratios less than one (the majority of genes).

These methods have already been used to demonstrate the occurrence of Darwinian (i.e., natural) molecular-level positive selection, resulting in amino acid differences in homologous proteins. Several groups have used such methods to document that a particular protein has evolved more rapidly than the neutral substitution rate, and thus supports the existence of Darwinian molecular-level positive selection.

### DETAILED DESCRIPTION OF THE INVENTION

The present invention utilizes comparative genomics to identify specific gene changes which are associated with, and thus may contribute to or be responsible for, commercially relevant traits in prokaryotes.

In a one embodiment, the methods described herein can be applied to identify the genes that control virulence traits in pathogenic bacteria. “Virulence,” as used herein, refers to the degree or ability of a pathogenic organism to cause disease. Although it has long been known that certain virulence factors for Bacillus anthracis are carried on two extra-chromosomal plasmids, both of which are required for full virulence, recent genomic analyses have made clear that an important set of genes for anthrax virulence lie in the main bacterial chromosome. When such virulence traits provide advantages to the pathogenic bacteria, the genes encoding such virulence traits are under selection pressure. This selection pressure is reflected in evolutionarily significant changes in genes encoding such virulence traits compared with homologous genes of less pathogenic, closely related prokaryotes. It has been found that only a few genes control pathogenic traits in some pathogenic bacteria. Some of these genes are encoded on plasmids and have been relatively easy to identify. However, other genes, encoded on bacterial chromosomes, have been harder to identify. The Ka/Ks and related analyses described herein can identify the genes controlling virulence traits or other selected traits of interest if those genes have undergone evolutionarily significant changes in the protein-coding region.

For any prokaryote of interest, genomic libraries can be constructed from the prokaryote and relevant closely related prokaryotes. As is described in U.S. Pat. No. 6,228,586, the libraries of each are “BLASTed” against each other to identify homologous polynucleotides. Alternatively, the skilled artisan can access commercially and/or publicly available genomic databases.

Next, a Ka/Ks or related analysis is conducted to identify selected genes that have rapidly evolved under selective pressure. These genes are then evaluated using standard in vitro and/or in vivo methods to determine if they play a role in the traits of commercial interest, such as pathogenesis. The genes of interest are used in assays to identify agents that may be useful as therapeutics because of their ability to inhibiting the pathogenic trait.

The practice of the present invention employs, unless otherwise indicated, conventional techniques of molecular biology, genetics and molecular evolution, which are within the skill of the art. Such techniques are explained fully in the literature, such as: “Molecular Cloning: A Laboratory Manual”, second edition (Sambrook et al., 1989); “Oligonucleotide Synthesis” (M. J. Gait, ed., 1984); “Current Protocols in Molecular Biology” (F. M. Ausubel et al., eds., 1987); “PCR: The Polymerase Chain Reaction”, (Mullis et al., eds., 1994); “Molecular Evolution”, (Li, 1997).

As used herein, a “polynucleotide” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified polynucleotides such as methylated and/or capped polynucleotides. The terms “polynucleotide” and “nucleotide sequence” are used interchangeably.

As used herein, a “gene” refers to a polynucleotide or portion of a polynucleotide comprising a sequence that encodes a protein. It is well understood in the art that a gene also comprises non-coding sequences, such as 5′ and 3′ flanking sequences (such as promoters, enhancers, repressors, and other regulatory sequences) as well as introns.

The terms “polypeptide,” “peptide,” and “protein” are used interchangeably herein to refer to polymers of amino acids of any length. These terms also include proteins that are post-translationally modified through reactions that include glycosylation, acetylation and phosphorylation.

The term “commercially relevant trait” is used herein to refer to traits that exist in prokaryote species whose analysis could provide information (e.g., physical or biochemical data) relevant to the development of agents that can modulate the polypeptide responsible for the trait. The commercially relevant trait can be unique, enhanced or altered relative to a closely related prokaryote. By “altered,” it is meant that the relevant trait differs qualitatively or quantitatively from traits observed in the closely related prokaryote.

The term “Ka/Ks-type methods” means methods that evaluate differences, frequently (but not always) shown as a ratio, between the number of nonsynonymous substitutions and synonymous substitutions in homologous genes (including the more rigorous methods that determine non-synonymous and synonymous sites). These methods are designated using several systems of nomenclature, including but not limited to Ka/Ks, d_{N}:d_{S}, D_{N}/D_{S}.

The terms “evolutionarily significant change” and “adaptive evolutionary change” refer to one or more nucleotide or peptide sequence change(s) between two organisms, species, subspecies, varieties, cultivars and/or strains that may be attributed to a positive selective pressure. One method for determining the presence of an evolutionarily significant change is to apply a Ka/Ks-type analytical method, such as to measure a Ka/Ks ratio. Typically, a Ka/Ks ratio at least about 1.0, in some embodiments at least about 1.25, in some embodiments at least about 1.5 and in some embodiments at least about 2.0 indicates the action of positive selection and is considered to be an evolutionarily significant change.

The term “positive evolutionarily significant change” means an evolutionarily significant change in a particular organism, species, subspecies, variety, cultivar or strain that results in an adaptive change that is positive as compared to other related organisms. An example of a positive evolutionarily significant change is a change that has resulted in increased virulence in pathogenic bacteria.

The term “resistant” means that an organism exhibits an ability to avoid, or diminish the extent of, a disease condition and/or development of the disease, such as when compared to non-resistant organisms.

The term “susceptibility” means that an organism fails to avoid, or diminish the extent of, a disease condition and/or development of the disease condition, such as when compared to an organism that is known to be resistant.

It is understood that resistance and susceptibility vary from individual to individual, and that, for purposes of this invention, these terms also apply to a group of individuals within a species, and comparisons of resistance and susceptibility generally refer to overall, average differences between species, although intra-specific comparisons may be used.

The term “homologous” or “homologue” or “ortholog” is known and well understood in the art and refers to related sequences that share a common ancestor and is determined based on degree of sequence identity. These terms describe the relationship between a gene found in one species, subspecies, variety, cultivar or strain and the corresponding or equivalent gene in another species, subspecies, variety, cultivar or strain. For purposes of this invention homologous sequences are compared. “Homologous sequences” or “homologues” or “orthologs” are thought, believed, or known to be functionally related. A functional relationship may be indicated in any one of a number of ways, including, but not limited to, (a) degree of sequence identity; (b) same or similar biological function. In some embodiments, both (a) and (b) are indicated. The degree of sequence identity may vary, but in some embodiments is at least 50% (when using standard sequence alignment programs known in the art), in some embodiments at least 60%, in other embodiments at least about 75%, and in other embodiments at least about 85%. Homology can be determined using software programs readily available in the art, such as those discussed in Current Protocols in Molecular Biology (F. M. Ausubel et al., eds., 1987) Supplement 30, section 7.718, Table 7.71. Exemplary alignment programs are MacVector (Oxford Molecular Ltd, Oxford, U.K.) and ALIGN Plus (Scientific and Educational Software, Pennsylvania). Another alignment program is Sequencher (Gene Codes, Ann Arbor, Mich.), using default parameters.

The term “nucleotide change” refers to nucleotide substitution, deletion, and/or insertion, as is well understood in the art.

The term “agent”, as used herein, means a biological or chemical compound such as a simple or complex organic or inorganic molecule, a peptide, a protein or an oligonucleotide that modulates the function of a polynucleotide or polypeptide. A vast array of compounds can be synthesized, for example oligomers, such as oligopeptides and oligonucleotides, and synthetic organic and inorganic compounds based on various core structures, and these are also included in the term “agent”. In addition, various natural sources can provide compounds for screening, such as plant or animal extracts, and the like. Compounds can be tested singly or in combination with one another.

The term “to modulate function” of a polynucleotide or a polypeptide means that the function of the polynucleotide or polypeptide is altered when compared to not adding an agent. Modulation may occur on any level that affects function. A polynucleotide or polypeptide function may be direct or indirect, and measured directly or indirectly.

A “function of a polynucleotide” includes, but is not limited to, replication; translation; expression pattern(s). A polynucleotide function also includes functions associated with a polypeptide encoded within the polynucleotide. For example, an agent which acts on a polynucleotide and affects protein expression, conformation, folding (or other physical characteristics), binding to other moieties (such as ligands), activity (or other functional characteristics), regulation and/or other aspects of protein structure or function is considered to have modulated polynucleotide function.

A “function of a polypeptide” includes, but is not limited to, conformation, folding (or other physical characteristics), binding to other moieties (such as ligands), activity (or other functional characteristics), and/or other aspects of protein structure or functions. For example, an agent that acts on a polypeptide and affects its conformation, folding (or other physical characteristics), binding to other moieties (such as ligands), activity (or other functional characteristics), and/or other aspects of protein structure or functions is considered to have modulated polypeptide function. The ways that an effective agent can act to modulate the function of a polypeptide include, but are not limited to 1) changing the conformation, folding or other physical characteristics; 2) changing the binding strength to its natural ligand or changing the specificity of binding to ligands; and 3) altering the activity of the polypeptide.

The term “target site” means a location in a polypeptide which can be a single amino acid and/or is a part of, a structural and/or functional motif, e.g., a binding site, a dimerization domain, or a catalytic active site. Target sites may be useful for direct or indirect interaction with an agent, such as a therapeutic agent.

The term “molecular difference” includes any structural and/or functional difference. Methods to detect such differences, as well as examples of such differences, are described herein.

A “functional effect” is a term well known in the art, and means any effect which is exhibited on any level of activity, whether direct or indirect.

The term “pathogenic” or “pathogenesis” refers to causing disease or resulting in the development of disease.

A “pathogenic trait” is a trait that results in disease or the development of disease.

A “virulence factor” is a gene or protein of a pathogenic organism that is associated with the pathogenicity of the organism.

General Procedures Known in the Art

For the purposes of this invention, the source of the polynucleotide from the prokaryote can be any suitable source, e.g., genomic sequences or cDNA sequences. In some embodiments, genomic sequences are compared. Genomic sequences can be obtained from available private, public and/or commercial databases such as those described herein. These databases serve as repositories of the molecular sequence data generated by ongoing research efforts. Alternatively, sequences may be obtained from, for example, sequencing of genomic DNA is prokaryote cells, or after PCR amplification, according to methods well known in the art.

General Methods of the Invention

The general method of the invention is as follows. Briefly, nucleotide sequences are obtained from a prokaryote and a closely related prokaryote. The nucleotide sequences are compared to one another to identify sequences that are homologous. The homologous sequences are analyzed to identify those that have nucleic acid sequence differences between the prokaryote and closely related prokaryote. Then molecular evolution analysis is conducted to evaluate quantitatively and qualitatively the evolutionary significance of the differences. For genes that have been positively selected, outgroup analysis can be done to identify those genes that have been positively selected in the prokaryote or the closely related prokaryote. Next, the sequence is characterized in terms of molecular/genetic identity and biological function. Finally, the information can be used to identify agents that can modulate the biological function of the gene or the polypeptide encoded by the gene.

The general methods of the invention entail comparing protein-coding nucleotide sequences of closely related prokaryotes. Bioinformatics is applied to the comparison and sequences are selected that contain a nucleotide change or changes that is/are evolutionarily significant change(s). The invention enables the identification of genes that have evolved to confer some evolutionary advantage and the identification of the specific evolved changes.

Any appropriate alignment mechanism for completing this comparison is contemplated by this invention. Alignment may be performed manually or by software (examples of suitable alignment programs are known in the art). In some embodiments, protein-coding sequences from a prokaryote are compared to the closely related prokaryote sequences via database searches, e.g., BLAST searches. The high scoring “hits,” i.e., sequences that show a significant similarity after BLAST analysis, will be retrieved and analyzed. Sequences showing a significant similarity can be those having at least about 60%, at least about 75%, at least about 80%, at least about 85%, or at least about 90% sequence identity. In some embodiments, sequences showing greater than about 80% identity are further analyzed. The homologous sequences identified via database searching can be aligned in their entirety using sequence alignment methods and programs that are known and available in the art, such as the commonly used simple alignment program CLUSTAL V by Higgins et al. (1992) CABIOS 8:189-191.

Alternatively, the sequencing and homology comparison of sequences between the a prokaryote and a closely related prokaryote may be performed simultaneously by using the sequencing chip technology. See, for example, Rava et al. U.S. Pat. No. 5,545,531.

The aligned sequences are analyzed to identify nucleotide sequence differences at particular sites. Again, any suitable method for achieving this analysis is contemplated by this invention. If there are no nucleotide sequence differences, the sequence is not usually further analyzed. The detected sequence changes are generally, and in some embodiments, initially checked for accuracy. In some embodiments, the initial checking comprises performing one or more of the following steps, any and all of which are known in the art: (a) finding the points where there are changes between the prokaryote sequences; (b) checking the sequence fluorogram (chromatogram) to determine if the bases that appear unique to the prokaryote or the closely related prokaryote correspond to strong, clear signals specific for the called base; (c) checking the hits to see if there is more than one prokaryote sequence that corresponds to a sequence change. Such changes are examined using database information and the genetic code to determine whether these nucleotide sequence changes result in a change in the amino acid sequence of the encoded protein. As the definition of “nucleotide change” makes clear, the present invention encompasses at least one nucleotide change, whether a substitution, a deletion or an insertion, in a protein-coding polynucleotide sequence of a prokaryote as compared to a corresponding sequence from a closely related prokaryote. In some embodiments, the change is a nucleotide substitution. In some embodiments, more than one substitution is present in the identified sequence and is subjected to molecular evolution analysis.

Any of several different molecular evolution analyses or Ka/Ks-type methods can be employed to evaluate quantitatively and qualitatively the evolutionary significance of the identified nucleotide changes between prokaryote gene sequences and those of corresponding closely related prokaryotes. Kreitman and Akashi (1995) Annu. Rev. Ecol. Syst. 26:403-422; Li, Molecular Evolution, Sinauer Associates, Sunderland, Mass., 1997. For example, positive selection on proteins (i.e., molecular-level adaptive evolution) can be detected in protein-coding genes by pairwise comparisons of the ratios of nonsynonymous nucleotide substitutions per nonsynonymous site (Ka) to synonymous substitutions per synonymous site (Ks) (Li et al., 1985; Li, 1993). Any comparison of Ka and Ks may be used, although it is particularly convenient and most effective to compare these two variables as a ratio. Sequences are identified by exhibiting a statistically significant difference between Ka and Ks using standard statistical methods.

In some embodiments, the Ka/Ks analysis by Li et al. is used to carry out the present invention, although other analysis programs that can detect positively selected genes between species can also be used. Li et al. (1985) Mol. Biol. Evol. 2:150-174; Li (1993); see also J. Mol. Evol. 36:96-99; Messier and Stewart (1997) Nature 385:151-154; Nei (1987) Molecular Evolutionary Genetics (New York, Columbia University Press). The Ka/Ks method, which comprises a comparison of the rate of non-synonymous substitutions per non-synonymous site with the rate of synonymous substitutions per synonymous site between homologous protein-coding region of genes in terms of a ratio, is used to identify sequence substitutions that may be driven by adaptive selections as opposed to neutral selections during evolution. A synonymous (“silent”) substitution is one that, owing to the degeneracy of the genetic code, makes no change to the amino acid sequence encoded; a non-synonymous substitution results in an amino acid replacement. The extent of each type of change can be estimated as Ka and Ks, respectively, the numbers of synonymous substitutions per synonymous site and non-synonymous substitutions per non-synonymous site. Calculations of Ka/s may be performed manually or by using software. An example of a suitable program is MEGA (Molecular Genetics Institute, Pennsylvania State University).

For the purpose of estimating Ka and Ks, either complete or partial protein-coding sequences are used to calculate total numbers of synonymous and non-synonymous substitutions, as well as non-synonymous and synonymous sites. The length of the polynucleotide sequence analyzed can be any appropriate length. In some embodiments, the entire coding sequence is compared, in order to determine any and all significant changes. Publicly available computer programs, such as Li93 (Li (1993) J. Mol. Evol. 36:96-99) or INA, can be used to calculate the Ka and Ks values for all pairwise comparisons. This analysis can be further adapted to examine sequences in a “sliding window” fashion such that small numbers of important changes are not masked by the whole sequence. “Sliding window” refers to examination of consecutive, overlapping subsections of the gene (the subsections can be of any length).

The comparison of non-synonymous and synonymous substitution rates is represented by the Ka/Ks ratio. Ka/Ks has been shown to be a reflection of the degree to which adaptive evolution has been at work in the sequence under study. Full length or partial segments of a coding sequence can be used for the Ka/Ks analysis. The higher the Ka/Ks ratio, the more likely that a sequence has undergone adaptive evolution and the non-synonymous substitutions are evolutionarily significant. See, for example, Messier and Stewart (1997). In some embodiments, the Ka/Ks ratio is at least about 1.0, in some embodiments at least about 1.25, in some embodiments at least about 1.50, or in some embodiments at least about 2.00. In some embodiments, statistical analysis is performed on all elevated Ka/Ks ratios, including, but not limited to, standard methods such as Student's t-test and likelihood ratio tests described by Yang (1998) Mol. Biol. Evol. 37:441-456.

For a pairwise comparison of homologous sequences, Ka/Ks ratios significantly greater than unity strongly suggest that positive selection has fixed greater numbers of amino acid replacements than can be expected as a result of chance alone, and is in contrast to the commonly observed pattern in which the ratio is less than or equal to one. Ratios less than one generally signify the role of negative, or purifying selection: there is strong pressure on the primary structure of functional, effective proteins to remain unchanged.

All methods for calculating Ka/Ks ratios are based on a pairwise comparison of the number of nonsynonymous substitutions per nonsynonymous site to the number of synonymous substitutions per synonymous site for the protein-coding regions of homologous genes from the prokaryote and the closely related prokaryote. Each method implements different corrections for estimating “multiple hits” (i.e., more than one nucleotide substitution at the same site). Each method also uses different models for how DNA sequences change over evolutionary time. Thus, in some embodiments, a combination of results from different algorithms is used to increase the level of sensitivity for detection of positively-selected genes and confidence in the result.

In some embodiments, Ka/Ks ratios should be calculated for orthologous gene pairs, as opposed to paralogous gene pairs (i.e., a gene which results from speciation, as opposed to a gene that is the result of gene duplication) Messier and Stewart (1997). This distinction may be made by performing additional comparisons with other closely related prokaryotes, which allows for phylogenetic tree-building. Orthologous genes when used in tree-building will yield the known “species tree”, i.e., will produce a tree that recovers the known biological tree. In contrast, paralogous genes will yield trees which will violate the known biological tree.

It is understood that the methods described herein could lead to the identification of polynucleotide sequences that are functionally related to the protein-coding sequences. Such sequences may include, but are not limited to, non-coding sequences or coding sequences that do not encode proteins. These related sequences can be, for example, physically adjacent to the protein-coding sequences in the genome, such as introns or 5′- and 3′-flanking sequences (including control elements such as promoters and enhancers). These related sequences may be obtained via searching available public, private and/or commercial genome databases or, alternatively, by screening and sequencing the organism's genomic library with a protein-coding sequence as probe. Methods and techniques for obtaining non-coding sequences using related coding sequence are well known for one skilled in the art.

The evolutionarily significant nucleotide changes, which are detected by molecular evolution analysis such as the Ka/Ks analysis, can be further assessed for their unique occurrence in the prokaryote or the extent to which these changes are unique in the prokaryote. For example, the identified changes in a gene of a pathogenic prokaryote can be tested for presence/absence in other sequences of related species, subspecies or other organisms closely related to the pathogenic prokaryote. This comparison (“outgroup analysis”) permits the determination of whether the positively selected gene is positively selected for the prokaryote.

The sequences with at least one evolutionarily significant change between a prokaryote and a closely related prokaryote can be used as primers for PCR analysis of other protein-coding sequences, and resulting polynucleotides are sequenced to see whether the same change is present in other closely related prokaryotes. These comparisons allow further discrimination as to whether the adaptive evolutionary changes are unique to the prokearyote lineage as compared to closely related prokaryotes or vice versa. A nucleotide change that is detected in a prokaryote but not other closely related prokaryotes more likely represents an adaptive evolutionary change in the prokaryote. Alternatively, a nucleotide change that is detected in a closely related prokaryote, but not in the prokaryote likely represents an adaptive evolutionary change in the closely related prokaryote. Other closely related prokaryotes used for comparison can be selected based on their phylogenetic relationships with the prokaryote. Statistical significance of such comparisons may be determined using established available programs, e.g., t-test as used by Messier and Stewart (1997) Nature 385:151-154. Those genes showing statistically high Ka/Ks ratios are very likely to have undergone adaptive evolution.

Sequences with significant changes can be used as probes in genomes from different prokaryote populations to see whether the sequence changes are shared by more than one prokaryote population. Gene sequences can be obtained from databases or, alternatively, from direct sequencing of PCR-amplified DNA from a number of diverse prokaryote populations. The presence of the identified changes in different prokaryote populations would further indicate the evolutionary significance of the changes.

Using the techniques of the present invention, heretofore unknown evolutionarily significant genes in B. anthracis, have been discovered as detailed in Example 2. K_{A}/K_{S} analysis, performed as described in Example 2 between B. cereus and B. anthracis (strain Ames) indicates an evolutionarily significant changes as shown in Table 1. These genes have been positively selected.

Sequences with significant changes between species can be further characterized in terms of their molecular/genetic identities and biological functions, using methods and techniques known to those of ordinary skill in the art. For example, the sequences can be located genetically and physically within the organism's genome using publicly available bio-informatics programs. The newly identified significant changes within the nucleotide sequence may suggest a potential role of the gene in the organism's evolution and a potential association with unique, enhanced or altered functional capabilities. The putative gene with the identified sequences may be further characterized by, for example, homologue searching. Shared homology of the putative gene with a known gene may indicate a similar biological role or function. Another exemplary method of characterizing a putative gene sequence is on the basis of known sequence motifs. Certain sequence patterns are known to code for regions of proteins having specific biological characteristics such as signal sequences, DNA binding domains, or transmembrane domains.

As another exemplary method of sequence characterization, the functional roles of the identified nucleotide sequences with evolutionarily significant changes can be assessed by conducting functional assays for different alleles of an identified gene in the prokaryote.

As another exemplary method of sequence characterization, the use of computer programs allows modeling and visualizing the three-dimensional structure of the homologous proteins from a prokaryote and a closely related prokaryote. Specific, exact knowledge of which amino acids have been replaced in the prokaryote protein(s) allows detection of structural changes that may be associated with functional differences. Thus, use of modeling techniques is closely associated with identification of functional roles discussed in the previous paragraph. The use of individual or combinations of these techniques constitutes part of the present invention.

The sequences identified by the methods described herein can be used to identify agents that are useful in modulating unique, enhanced or altered functional capabilities of a prokaryote. These methods employ, for example, screening techniques known in the art, such as in vitro systems or cell-based expression systems.

A prokaryote's gene identified by the subject method can be used to identify homologous genes in other species.

The present invention also provides a method of detecting a virulence-related gene in a prokaryote comprising: a) contacting the gene or a portion thereof greater than 12 nucleotides, or in some cases greater than 30 nucleotides in length with a preparation of genomic DNA from the prokaryote under hybridization conditions providing detection of nucleic acid molecule sequences having about 50% or greater sequence identity to the a nucleic acid molecule selected from the group consisting of SEQ ID NO:1, SEQ ID NO:7, SEQ ID. NO:13, SEQ ID NO:19, SEQ ID NO:25, and SEQ ID NO:31, and b) detecting hybridization, whereby a virulence-related gene may be identified.

The present invention also provides a method of isolating a virulence-related gene, comprising a) providing a preparation of bacterial DNA or a recombinant bacterial library; b) contacting the preparation or library with a detectably-labelled virulence-related oligonucleotide under hybridization conditions providing detection of genes having 50% or greater sequence identity; and c) isolating a virulence-related gene by its association with the detectable label.

The present invention also provides a method of isolating a virulence-related gene from bacterial cell DNA comprising a) providing a sample of bacterial DNA; b) providing a pair of oligonucleotides having sequence homology to a conserved region of a virulence gene; c) combining the pair of oligonucleotides with the bacterial DNA sample under conditions suitable for polymerase chain reaction-mediated DNA amplification; and d) isolating the amplified virulence-related gene or fragment thereof.

The sequences identified by the methods described herein can be used to identify agents that are useful in modulating domesticated organism-unique, enhanced or altered functional capabilities and/or correcting defects in these capabilities using these sequences. These methods employ, for example, screening techniques known in the art, such as in vitro systems, cell-based expression systems and transgenic animals and bacterials. The approach provided by the present invention not only identifies rapidly evolved genes, but indicates modulations that can be made to the protein that may not be too toxic because they exist in another species.

The present invention also provides a method of producing an virulence-related polypeptide comprising: a) providing a cell transfected with a polynucleotide encoding an virulence-related polypeptide positioned for expression in the cell; b) culturing the transfected cell under conditions for expressing the polynucleotide; and c) isolating the virulence-related polypeptide.

The sequences identified by the methods described herein can be used to identify agents that are useful in modulating domesticated organism-unique, enhanced or altered functional capabilities and/or correcting defects in these capabilities using these sequences. These methods employ, for example, screening techniques known in the art, such as in vitro systems, cell-based expression systems and transgenic animals and bacterials. The approach provided by the present invention not only identifies rapidly evolved genes, but indicates modulations that can be made to the protein that may not be too toxic because they exist in another species.

One embodiment of the present invention is an isolated virulence-related polypeptide. As used herein, a virulence-related polypeptide, in one embodiment, is a polypeptide that is related to (i.e., bears structural similarity to) the B. anthracis polypeptides having the sequences depicted in SEQ ID NO:3, SEQ ID NO:9, SEQ ID NO:15, SEQ ID NO:21, SEQ ID NO:27, and SEQ ID NO:33. The original identification of such polypeptides is detailed in the Examples. In one embodiment, a virulence-related polypeptide is encoded by a polynucleotide that hybridizes under stringent hybridization conditions to at a gene encoding an B. anthracis virulence-related polypeptide (i.e., a B. anthracis gene). It is to be noted that the term “a” or “an” entity refers to one or more of that entity; for example, a gene refers to one or more genes or at least one gene. As such, the terms “a” (or “an”), “one or more” and “at least one” can be used interchangeably herein. It is also to be noted that the terms “comprising,” “including,” and “having” can be used interchangeably.

As used herein, stringent hybridization conditions refer to standard hybridization conditions under which polynucleotides, including oligonucleotides, are used to identify molecules having similar nucleic acid sequences. Such standard conditions are disclosed, for example, in Sambrook et al., MOLECULAR CLONING: A LABORATORY MANUAL, Cold Spring Harbor Labs Press, 1989. Examples of such conditions are provided in the Examples section of the present application.

As used herein, a B. anthracis virulence-related gene includes all nucleic acid sequences related to a natural B. anthracis virulence-related gene such as regulatory regions that control production of the B. anthracis virulence-related polypeptide encoded by that gene (such as, but not limited to, transcription, translation or post-translation control regions) as well as the coding region itself. In one embodiment, a B. anthracis virulence-related gene includes the nucleic acid sequence SEQ ID NO: 1, SEQ ID NO:7, SEQ ID.NO:13, SEQ ID NO:19, SEQ ID NO:25, and SEQ ID NO:31. These nucleic acid sequence represent the deduced sequence of a polynucleotide, the identification of which is disclosed in the Examples. It should be noted that since nucleic acid sequencing technology is not entirely error-free, SEQ ID NO:1 (as well as other sequences presented herein), at best, represents an apparent nucleic acid sequence of the polynucleotide encoding an B. anthracis virulence-related polypeptide of the present invention.

In another embodiment, a B. anthracis virulence-related gene can be an allelic variant that includes a similar but not identical sequence to SEQ ID NO:1, SEQ ID NO:7, SEQ ID. NO:13, SEQ ID NO:19, SEQ ID NO:25, or SEQ ID NO:31. An allelic variant of a B. anthracis virulence-related gene including any of SEQ ID NO:1, SEQ ID NO:7, SEQ ID. NO:13, SEQ ID NO:19, SEQ ID NO:25, or SEQ ID NO:31 is a locus (or loci) in the genome whose activity is concerned with the same biochemical or developmental processes, and/or a gene that that occurs at essentially the same locus as the gene including the SEQ ID NO:, but which, due to natural variations caused by, for example, mutation or recombination, has a similar but not identical sequence. Because genomes can undergo rearrangement, the physical arrangement of alleles is not always the same. Allelic variants typically encode polypeptides having similar activity to that of the polypeptide encoded by the gene to which they are being compared. Allelic variants can also comprise alterations in the 5′ or 3′ untranslated regions of the gene (e.g., in regulatory control regions). Allelic variants are well known to those skilled in the art and would be expected to be found within a given bacteria or strain.

According to the present invention, an isolated, or biologically pure, polypeptide, is a polypeptide that has been removed from its natural milieu. As such, “isolated” and “biologically pure” do not necessarily reflect the extent to which the polypeptide has been purified. An isolated virulence-related polypeptide of the present invention can be obtained from its natural source, can be produced using recombinant DNA technology or can be produced by chemical synthesis. A virulence-related polypeptide of the present invention may be identified by its ability to perform the function of natural virulence-related in a functional assay. By “natural virulence-related polypeptide,” it is meant the full length virulence-related polypeptide of B. anthracis. The phrase “capable of performing the function of a natural virulence-related polypeptidein a functional assay” means that the polypeptide has at least about 10% of the activity of the natural polypeptide in the functional assay. In other embodiments, the virulence-related polypeptide has at least about 20% of the activity of the natural polypeptide in the functional assay. In other embodiments, the virulence-related polypeptide has at least about 30% of the activity of the natural polypeptide in the functional assay. In yet other embodiments, the virulence-related polypeptide has at least about 40% of the activity of the natural polypeptide in the functional assay. In still other embodiments, the virulence-related polypeptide has at least about 50% of the activity of the natural polypeptide in the functional assay. In other embodiments, the polypeptide has at least about 60% of the activity of the natural polypeptide in the functional assay. In other embodiments, the polypeptide has at least about 70% of the activity of the natural polypeptide in the functional assay. In other embodiments, the polypeptide has at least about 80% of the activity of the natural polypeptide in the functional assay. In other embodiments, the polypeptide has at least about 90% of the activity of the natural polypeptide in the functional assay. Examples of functional assays include antibody-binding assays, virulence-increasing assays or virulence-decreasing assays, as detailed elsewhere in this specification.

As used herein, an isolated virulence-related polypeptide can be a full-length polypeptide or any homologue of such a polypeptide. Examples of virulence-related homologues include virulence-related polypeptides in which amino acids have been deleted (e.g., a truncated version of the polypeptide, such as a peptide), inserted, inverted, substituted and/or derivatized (e.g., by glycosylation, phosphorylation, acetylation, myristylation, prenylation, palmitoylation, amidation and/or addition of glycerophosphatidyl inositol) such that the homolog has the natural virulence-related polypeptide activity.

In one embodiment, when the homologue is administered to an animal as an immunogen, using techniques known to those skilled in the art, the animal will produce a humoral and/or cellular immune response against at least one epitope of a natural virulence-related polypeptide. virulence-related polypeptide homologues can also be selected by their ability to perform the function of virulence-related polypeptide in a functional assay.

Virulence-related polypeptide homologues can be the result of natural allelic variation or natural mutation. Virulence-related polypeptide homologues of the present invention can also be produced using techniques known in the art including, but not limited to, direct modifications to the polypeptide or modifications to the gene encoding the polypeptide using, for example, classic or recombinant DNA techniques to effect random or targeted mutagenesis.

In accordance with the present invention, a mimetope refers to any compound that is able to mimic the ability of an isolated virulence-related polypeptide of the present invention to perform the function of an virulence-related polypeptide of the present invention in a functional assay. Examples of mimetopes include, but are not limited to, anti-idiotypic antibodies or fragments thereof, that include at least one binding site that mimics one or more epitopes of an isolated polypeptide of the present invention; non-polypeptideaceous immunogenic portions of an isolated polypeptide (e.g., carbohydrate structures); and synthetic or natural organic molecules, including nucleic acids, that have a structure similar to at least one epitope of an isolated polypeptide of the present invention. Such mimetopes can be designed using computer-generated structures of polypeptides of the present invention. Mimetopes can also be obtained by generating random samples of molecules, such as oligonucleotides, peptides or other organic molecules, and screening such samples by affinity chromatography techniques using the corresponding binding partner.

The minimal size of a virulence-related polypeptide homologue of the present invention is a size sufficient to be encoded by a polynucleotide capable of forming a stable hybrid with the complementary sequence of a polynucleotide encoding the corresponding natural polypeptide. As such, the size of the polynucleotide encoding such a polypeptide homologue is dependent on nucleic acid composition and percent homology between the polynucleotide and complementary sequence as well as upon hybridization conditions per se (e.g., temperature, salt concentration, and formamide concentration). It should also be noted that the extent of homology required to form a stable hybrid can vary depending on whether the homologous sequences are interspersed throughout the polynucleotides or are clustered (i.e., localized) in distinct regions on the polynucleotides. The minimal size of such polynucleotides is typically at least about 12 to about 15 nucleotides in length if the polynucleotides are GC-rich and at least about 15 to about 17 bases in length if they are AT-rich. In some embodiments, the polynucleotide is at least 12 bases in length.

As such, the minimal size of a polynucleotide used to encode a virulence-related polypeptide homologue of the present invention is from about 12 to about 18 nucleotides in length. There is no limit, other than a practical limit, on the maximal size of such a polynucleotide in that the polynucleotide can include a portion of a gene, an entire gene, or multiple genes, or portions thereof. Similarly, the minimal size of a virulence-related polypeptide homologue of the present invention is from about 4 to about 6 amino acids in length, with sizes depending on whether a full-length, fusion, multivalent, or functional portions of such polypeptides are desired. In some embodiments, the polypeptide is at least 30 amino acids in length.

Any bacterial virulence-related polypeptide is a suitable polypeptide of the present invention. Suitable bacteria from which to identify and isolate virulence-related polypeptides (including isolation of the natural polypeptide or production of the polypeptide by recombinant or synthetic techniques) include any pathogenic bacteria having a non-pathogenic relative, including, but not limited to Staphyococcus aureus and other Staphyococcus spp., Pseudomonas aeruginosa, and other Pseudomonas spp., Yersinia pestis and other Yersinia spp., Legionella pneumoniae and other Legionella spp., Vibrio cholerae and other Vibrio spp., Neisseria spp., Streptococcus. pyogenes, and other Group A, Group B, and Group G Streptococcus spp.

One virulence-related polypeptide of the present invention is a molecue that when expressed or modulated in a bacteria, is capable of increasing the virulence of the bacteria. In some embodiments, for example, if the polypeptide is to be used as an antibacterial drug, a polypeptide of the present invention is capable of decreasing the virulence of the bacteria.

One embodiment of the present invention is a fusion polypeptide that includes a virulence-related polypeptide-containing domain attached to a fusion segment. Inclusion of a fusion segment as part of a virulence-related polypeptide of the present invention can enhance the polypeptide's stability during production, storage and/or use. Depending on the segment's characteristics, a fusion segment can also act as an immunopotentiator to enhance the immune response mounted by an animal immunized with an virulence-related polypeptide containing such a fusion segment. Furthermore, a fusion segment can function as a tool to simplify purification of a virulence-related polypeptide, such as to enable purification of the resultant fusion polypeptide using affinity chromatography. A suitable fusion segment can be a domain of any size that has the desired function (e.g., imparts increased stability, imparts increased immunogenicity to a polypeptide, and/or simplifies purification of a polypeptide). It is within the scope of the present invention to use one or more fusion segments. Fusion segments can be joined to amino and/or carboxyl termini of the virulence-related-containing domain of the polypeptide. Linkages between fusion segments and virulence-related-containing domains of fusion polypeptides can be susceptible to cleavage in order to enable straightforward recovery of the virulence-related-containing domains of such polypeptides. Fusion polypeptides may be produced by culturing a recombinant cell transformed with a fusion polynucleotide that encodes a polypeptide including the fusion segment attached to either the carboxyl and/or amino terminal end of a virulence-related-containing domain.

Fusion segments which may be used in the present invention include a glutathione binding domain; a metal binding domain, such as a poly-histidine segment capable of binding to a divalent metal ion; an immunoglobulin binding domain, such as Polypeptide A, Polypeptide G, T cell, B cell, Fc receptor or complement polypeptide antibody-binding domains; a sugar binding domain such as a maltose binding domain from a maltose binding polypeptide; and/or a “tag” domain (e.g., at least a portion of β-galactosidase, a strep tag peptide, other domains that can be purified using compounds that bind to the domain, such as monoclonal antibodies). Additional fusion segments include metal binding domains, such as a poly-histidine segment; a maltose binding domain; a strep tag peptide.

One B. anthracis virulence-related polypeptide of the present invention is a polypeptide encoded by a B. anthracis polynucleotide that hybridizes under stringent hybridization conditions with complements of polynucleotides represented by SEQ ID NO:1, SEQ ID NO:7, SEQ ID. NO:13, SEQ ID NO:19, SEQ ID NO:25, and/or SEQ ID NO:31. Such a virulence-related polypeptide is encoded by a polynucleotide that hybridizes under stringent hybridization conditions with a polynucleotide having nucleic acid sequence SEQ ID NO:1, SEQ ID NO:7, SEQ ID. NO:13, SEQ ID NO:19, SEQ ID NO:25, and/or SEQ ID NO:31.

B. anthracis virulence-related polynucleotide SEQ ID NO:1 suggests an open reading frame from about nucleotide 1 to about nucleotide 1161 of SEQ ID NO:1. The reading frame encodes a B. anthracis virulence-related polypeptide of about 386 amino acids, the deduced amino acid sequence of which is represented herein as SEQ ID NO:3.

Similarly, translation of B. anthracis polynucleotide SEQ ID NO:7 suggests an open reading frame from about nucleotide 1 to about nucleotide 2331 of SEQ ID NO:7, and encodes a polypeptide of about 776 amino acids represented herein as SEQ ID NO:9.

Similarly, translation of B. anthracis polynucleotide SEQ ID NO:13 suggests an open reading frame from about nucleotide 1 to about nucleotide 354 of SEQ ID NO:13, and encodes a polypeptide of about 117 amino acids represented herein as SEQ ID NO:15.

Similarly, translation of B. anthracis polynucleotide SEQ ID NO:19 suggests an open reading frame from about nucleotide 1 to about nucleotide 615 of SEQ ID NO:19, and encodes a polypeptide of about 204 amino acids represented herein as SEQ ID NO:21.

Similarly, translation of B. anthracis polynucleotide SEQ ID NO:25 suggests an open reading frame from about nucleotide 1 to about nucleotide 255 of SEQ ID NO:25, and encodes a polypeptide of about 84 amino acids represented herein as SEQ ID NO:27.

Similarly, translation of B. anthracis polynucleotide SEQ ID NO:31 suggests an open reading frame from about nucleotide 1 to about nucleotide 273 of SEQ ID NO:31, and encodes a polypeptide of about 90 amino acids represented herein as SEQ ID NO:33.

Comparison of the various B. anthracis virulence-related nucleic acid sequences and amino acid sequences indicates that this species possesses genes and polypeptides similar to those found in other prokaryotes. For example, based on homology with known proteins, SEQ ID NO:3 can be classified as a putative endopeptidase. Based on homology with known proteins, SEQ ID NO:9 can be classified as a hydrogenase maturation protein. Based on homology with known proteins, SEQ ID NO:21 can be classified as a putative cell wall endopeptidase of family M23/M37. Based on homology with known proteins, SEQ ID NO:15, 27, and 33 can not be classified. Thus, SEQ ID NO:15, 27, and 33 represent previously unknown virulence-related polypeptides.

Finding some degree of identity between B. anthracis virulence-related nucleic acid sequences and amino acid sequences and those of other bacteria supports the ability to obtain any virulence-related polypeptide and polynucleotide given the polypeptide and nucleic acid sequences disclosed herein.

These bacterial virulence-related polypeptides, and the polynucleotides that encode them, represent compounds with utility as targets for antibacterial drugs.

Some bacterial virulence-related polypeptides of the present invention include polypeptides comprising amino acid sequences that are at least about 30%, in some embodiments at least about 50%, in other embodiments at least about 75%, in still other embodiments at least about 80%, in still other embodiments at least about 85%, in still other embodiments at least about 90%, and in still other embodiments at least about 95%, in still other embodiments at least about 98% identical to one or more of the amino acid sequences disclosed herein for B. anthracis virulence-related polypeptides of the present invention.

Some bacterial virulence-related polypeptides of the present invention include: polypeptides encoded by at least a portion of SEQ ID NO.1 and, as such, have amino acid sequences that include at least a portion of SEQ ID NO:3; polypeptides encoded by at least a portion of SEQ ID NO:7, and, as such, have amino acid sequences that include at least a portion of SEQ ID NO:9; polypeptides encoded by at least a portion of SEQ ID NO:13 and, as such, have amino acid sequences that include at least a portion of SEQ ID NO:15; polypeptides encoded by at least a portion of SEQ ID NO:19 and, as such, have amino acid sequences that include at least a portion of SEQ ID NO:21; polypeptides encoded by at least a portion of SEQ ID NO:25 and, as such, have amino acid sequences that include at least a portion of SEQ ID NO:27; polypeptides encoded by at least a portion of SEQ ID NO:31 and, as such, have amino acid sequences that include at least a portion of SEQ ID NO:33. As used herein, “at least a portion” of a polynucleotide or polypeptide means a portion having the minimal size characteristics of such sequences, as described above, or any larger fragment of the full length molecule, up to and including the full length molecule. For example, a portion of a polynucleotide may be 12 nucleotides, 13 nucleotides, 14 nucleotides, 15 nucleotides, and so on, going up to the full length polynucleotide. Similarly, a portion of a polypeptide may be 4 amino acids, 5 amino acids, 6 amino acids, 7 amino acids, and so on, going up to the full length polypeptide. The length of the portion to be used will depend on the particular application. As discussed above, a portion of a polynucleotide useful as hybridization probe may be as short as 12 nucleotides. A portion of a polypeptide useful as an epitope may be as short as 4 amino acids. A portion of a polypeptide that performs the function of the full-length polypeptide would generally be longer than 4 amino acids.

In some embodiments, bacterial virulence-related polypeptides of the present invention are polypeptides that include SEQ ID NO:3, SEQ ID NO:9, SEQ ID NO:15, SEQ ID NO:21, SEQ ID NO:27, and/or SEQ ID NO:33 (including, but not limited to the encoded polypeptides, full-length polypeptides, processed polypeptides, fusion polypeptides and multivalent polypeptides thereof) as well as polypeptides that are truncated homologues of polypeptides that include at least portions of the aforementioned SEQ ID NOs. Examples of methods to produce such polypeptides are known in the art.

One embodiment of the present invention is an isolated bacterial polynucleotide that hybridizes under stringent hybridization conditions with a B. anthracis virulence-related gene. The identifying characteristics of such genes are heretofore described. A polynucleotide of the present invention can include an isolated natural bacterial virulence-related gene or a homologue thereof, the latter of which is described in more detail below. A polynucleotide of the present invention can include one or more regulatory regions, full-length or partial coding regions, or combinations thereof. The minimal size of a polynucleotide of the present invention is the minimal size that can form a stable hybrid with one of the aforementioned genes under stringent hybridization conditions. Suitable bacteria are disclosed above.

In accordance with the present invention, an isolated polynucleotide is a polynucleotide that has been removed from its natural milieu (i.e., that has been subject to human manipulation). As such, “isolated” does not reflect the extent to which the polynucleotide has been purified. An isolated polynucleotide can include DNA, RNA, or derivatives of either DNA or RNA.

An isolated bacterial virulence-related polynucleotide of the present invention can be obtained from its natural source either as an entire (i.e., complete) gene or a portion thereof capable of forming a stable hybrid with that gene. An isolated bacterial virulence-related polynucleotide can also be produced using recombinant DNA technology (e.g., polymerase chain reaction (PCR) amplification, cloning) or chemical synthesis. Isolated bacterial virulence-related polynucleotides include natural polynucleotides and homologues thereof, including, but not limited to, natural allelic variants and modified polynucleotides in which nucleotides have been inserted, deleted, substituted, and/or inverted in such a manner that such modifications do not substantially interfere with the polynucleotide's ability to encode a virulence-related polypeptide of the present invention or to form stable hybrids under stringent conditions with natural gene isolates.

A bacterial virulence-related polynucleotide homologue can be produced using a number of methods known to those skilled in the art (see, for example, Sambrook et al., ibid.). For example, polynucleotides can be modified using a variety of techniques including, but not limited to, classic mutagenesis techniques and recombinant DNA techniques, such as site-directed mutagenesis, chemical treatment of a polynucleotide to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of polynucleotides and combinations thereof. Polynucleotide homologues can be selected from a mixture of modified nucleic acids by screening for the function of the polypeptide encoded by the nucleic acid (e.g., ability to elicit an immune response against at least one epitope of a virulence-related polypeptide, ability to increase virulence in a recombinant prokaryote containing a virulence-related gene) and/or by hybridization with a B. anthracis virulence-related gene.

An isolated polynucleotide of the present invention can include a nucleic acid sequence that encodes at least one bacterial virulence-related polypeptide of the present invention, examples of such polypeptides being disclosed herein. Although the phrase “polynucleotide” primarily refers to the physical polynucleotide and the phrase “nucleic acid sequence” primarily refers to the sequence of nucleotides on the polynucleotide, the two phrases can be used interchangeably, especially with respect to a polynucleotide, or a nucleic acid sequence, being capable of encoding a virulence-related polypeptide. As heretofore disclosed, bacterial virulence-related polypeptides of the present invention include, but are not limited to, polypeptides having full-length bacterial virulence-related coding regions, polypeptides having partial bacterial virulence-related coding regions, fusion polypeptides, multivalent protective polypeptides and combinations thereof.

At least certain polynucleotides of the present invention encode polypeptides that selectively bind to immune serum derived from an animal that has been immunized with a virulence-related polypeptide from which the polynucleotide was isolated.

A polynucleotide of the present invention, when expressed in a suitable prokaryote, is capable of increasing the virulence of the bacteria. As will be disclosed in more detail below, such a polynucleotide can be, or encode, an antisense RNA, a molecule capable of triple helix formation, a ribozyme, or other nucleic acid-based compound. In some embodiments, for example, if the polynucleotide is to be used as an antibacterial drug, a polynucleotide of the present invention is capable of decreasing the virulence of the bacteria.

One embodiment of the present invention is a bacterial virulence-related polynucleotide that hybridizes under stringent hybridization conditions to a virulence-related polynucleotide of the present invention, or to a homologue of such a virulence-related polynucleotide, or to the complement of such a polynucleotide. A polynucleotide complement of any nucleic acid sequence of the present invention refers to the nucleic acid sequence of the polynucleotide that is complementary to (i.e., can form a complete double helix with) the strand for which the sequence is cited. It is to be noted that a double-stranded nucleic acid molecule of the present invention for which a nucleic acid sequence has been determined for one strand, that is represented by a SEQ ID NO, also comprises a complementary strand having a sequence that is a complement of that SEQ ID NO. As such, polynucleotides of the present invention, which can be either double-stranded or single-stranded, include those polynucleotides that form stable hybrids under stringent hybridization conditions with either a given SEQ ID NO denoted herein and/or with the complement of that SEQ ID NO, which may or may not be denoted herein. Methods to deduce a complementary sequences are known to those skilled in the art. A virulence-related polynucleotide that includes a nucleic acid sequence having at least about 65 percent, in some embodiments at least about 70 percent, in other embodiments at least about 75 percent, in still other embodiments at least about 80 percent, in still other embodiments at least about 85 percent, in still other embodiments at least about 90 percent and in still other embodiments at least about 95 percent homology with the corresponding region(s) of the nucleic acid sequence encoding at least a portion of a virulence-related polypeptide may be used. A virulence-related polynucleotide capable of encoding at least a portion of a virulence-related polypeptide that naturally is present in bacteria may be used.

Some virulence-related polynucleotides of the present invention hybridize under stringent hybridization conditions with at least one of the following polynucleotides: SEQ ID NO:1, SEQ ID NO:7, SEQ ID. NO: 13, SEQ ID NO:19, SEQ ID NO:25, and/or SEQ ID NO:31, or to a homologue or complement of such polynucleotide.

Some polynucleotides of the present invention include at least a portion of nucleic acid sequence SEQ ID NO:1, SEQ ID NO:7, SEQ ID. NO:13, SEQ ID NO:19, SEQ ID NO:25, and/or SEQ ID NO:31 that is capable of hybridizing (i.e., that hybridizes under stringent hybridization conditions) to a B. anthracis virulence-related gene of the present invention, as well as a polynucleotide that is an allelic variant of any of those polynucleotides. Such polynucleotides can include nucleotides in addition to those included in the SEQ ID NOs, such as, but not limited to, a full-length gene, a full-length coding region, a polynucleotide encoding a fusion polypeptide, and/or a polynucleotide encoding a multivalent protective compound.

The present invention also includes polynucleotides encoding a polypeptide including at least a portion of SEQ ID NO:3, polynucleotides encoding a polypeptide having at least a portion of SEQ ID NO:9, polynucleotides encoding a polypeptide having at least a portion of SEQ ID NO:15, polynucleotides encoding a polypeptide having at least a portion of SEQ ID NO:21, polynucleotides encoding a polypeptide having at least a portion of SEQ ID NO:27, polynucleotides encoding a polypeptide having at least a portion of SEQ ID NO:33, including polynucleotides that have been modified to accommodate codon usage properties of the cells in which such polynucleotides are to be expressed.

Knowing the nucleic acid sequences of certain bacterial virulence-related polynucleotides of the present invention allows one skilled in the art to, for example, (a) make copies of those polynucleotides, (b) obtain polynucleotides including at least a portion of such polynucleotides (e.g., polynucleotides including full-length genes, full-length coding regions, regulatory control sequences, truncated coding regions), and (c) obtain virulence-related polynucleotides for other prokaryotes, particularly since, knowledge of B. anthracis virulence-related polynucleotides of the present invention enables the isolation of other polynucleotides. Such polynucleotides can be obtained in a variety of ways including screening appropriate expression libraries with antibodies of the present invention; traditional cloning techniques using oligonucleotide probes of the present invention to screen appropriate libraries or DNA; and PCR amplification of appropriate libraries or DNA using oligonucleotide primers of the present invention. Such libraries to screen or from which to amplify polynucleotides include libraries such as genomic DNA libraries, BAC libraries, YAC libraries, cDNA libraries prepared from isolated bacteria. Similarly, some DNA sources to screen or from which to amplify polynucleotides include bacterial genomic DNA. Techniques to clone and amplify genes are disclosed, for example, in Sambrook et al., ibid.

The present invention also includes polynucleotides that are oligonucleotides capable of hybridizing, under stringent hybridization conditions, with complementary regions of other, sometimes longer, polynucleotides of the present invention such as those comprising bacterial virulence-related genes or other bacterial virulence-related polynucleotides. Oligonucleotides of the present invention can be RNA, DNA, or derivatives of either. The minimal size of such oligonucleotides is the size required to form a stable hybrid between a given oligonucleotide and the complementary sequence on another polynucleotide of the present invention. Minimal size characteristics are disclosed herein. The size of the oligonucleotide must also be sufficient for the use of the oligonucleotide in accordance with the present invention. Oligonucleotides of the present invention can be used in a variety of applications including, but not limited to, as probes to identify additional polynucleotides, as primers to amplify or extend polynucleotides, as targets for expression analysis, as candidates for targeted mutagenesis and/or recovery, or in agricultural applications to alter virulence-related polypeptide production or activity. Such agricultural applications include the use of such oligonucleotides in, for example, antisense-, triplex formation-, ribozyme- and/or RNA drug-based technologies. The present invention, therefore, includes such oligonucleotides and methods to prepare antibacterials by use of one or more of such technologies.

The present invention also includes isolated antibodies capable of selectively binding to a virulence-related polypeptide of the present invention or to a mimetope thereof. Such antibodies are also referred to herein as anti-virulence-related polypeptide antibodies. Some antibodies of this embodiment include anti-B. anthracis virulence-related polypeptide antibodies.

Isolated antibodies are antibodies that have been removed from their natural milieu. The term “isolated” does not refer to the state of purity of such antibodies. As such, isolated antibodies can include anti-sera containing such antibodies, or antibodies that have been purified to varying degrees.

As used herein, the term “selectively binds to” refers to the ability of antibodies of the present invention to bind, in some embodiments, to specified polypeptides and mimetopes thereof of the present invention. Binding can be measured using a variety of methods known to those skilled in the art including immunoblot assays, immunoprecipitation assays, radioimmunoassays, enzyme immunoassays (e.g., ELISA), immunofluorescent antibody assays and immunoelectron microscopy; see, for example, Sambrook et al., ibid., and Harlow & Lane, 1990, ibid.

Antibodies of the present invention can be either polyclonal or monoclonal antibodies. Antibodies of the present invention include functional equivalents such as antibody fragments and genetically-engineered antibodies, including single chain antibodies, that are capable of selectively binding to at least one of the epitopes of the polypeptide or mimetope used to obtain the antibodies. Antibodies of the present invention also include chimeric antibodies that can bind to more than one epitope. Some antibodies are raised in response to polypeptides, or mimetopes thereof, that are encoded, at least in part, by a polynucleotide of the present invention.

A method to produce antibodies of the present invention includes (a) administering to an animal an effective amount of a polypeptide or mimetope thereof of the present invention to produce the antibodies and (b) recovering the antibodies. In another method, antibodies of the present invention are produced recombinantly using techniques as heretofore disclosed to B. anthracis virulence-related polypeptides of the present invention.

Antibodies of the present invention have a variety of potential uses that are within the scope of the present invention. For example, such antibodies can be used (a) as reagents in assays to detect expression of virulence-related polypeptides and/or (b) as tools to screen expression libraries and/or to recover desired polypeptides of the present invention from a mixture of polypeptides and other contaminants. Furthermore, antibodies of the present invention can be used to target cytotoxic agents to bacteria in order to directly kill such bacteria. Targeting can be accomplished by conjugating (i.e., stably joining) such antibodies to the cytotoxic agents using techniques known to those skilled in the art. Suitable cytotoxic agents are known to those skilled in the art. Suitable cytotoxic agents include, but are not limited to: double-chain polypeptides (i.e., toxins having A and B chains), such as diphtheria toxin, ricin toxin, Pseudomonas exotoxin, modeccin toxin, abrin toxin, and shiga toxin; single-chain toxins, such as pokeweed antiviral polypeptide, α-amanitin, and ribosome inhibiting polypeptides; and chemical toxins, such as melphalan, methotrexate, nitrogen mustard, doxorubicin and daunomycin. Some double-chain toxins are modified to include the toxic domain and translocation domain of the toxin but lack the toxin's intrinsic cell binding domain.

Screening Methods

The present invention also provides screening methods using the polynucleotides and polypeptides identified and characterized using the above-described methods. These screening methods are useful for identifying agents which may modulate the function(s) of the polynucleotides or polypeptides in a manner that would be useful for enhancing or diminishing a characteristic in a prokaryote. Generally, the methods entail contacting at least one agent to be tested a cell containing a polynucleotide sequence identified by the methods described above, or a preparation of the polypeptide encoded by such polynucleotide sequence, wherein an agent is identified by its ability to modulate function of either the polynucleotide sequence or the polypeptide. For example, an agent can be a compound that is applied as a therapeutic to treat humans or animals infected with a pathogenic prokaryote.

As used herein, the term “agent” means a biological or chemical compound such as a simple or complex organic or inorganic molecule, a peptide, a protein or an oligonucleotide. A vast array of compounds can be synthesized, for example oligomers, such as oligopeptides and oligonucleotides, and synthetic organic and inorganic compounds based on various core structures, and these are also included in the term “agent”. In addition, various natural sources can provide compounds for screening, such as plant or animal extracts, and the like. Compounds can be tested singly or in combination with one another.

To “modulate function” of a polynucleotide or a polypeptide means that the function of the polynucleotide or polypeptide is altered when compared to not adding an agent. Modulation may occur on any level that affects function. A polynucleotide or polypeptide function may be direct or indirect, and measured directly or indirectly. A “function” of a polynucleotide includes, but is not limited to, replication, translation, and expression pattern(s). A polynucleotide function also includes functions associated with a polypeptide encoded within the polynucleotide. For example, an agent which acts on a polynucleotide and affects protein expression, conformation, folding (or other physical characteristics), binding to other moieties (such as ligands), activity (or other functional characteristics), regulation and/or other aspects of protein structure or function is considered to have modulated polynucleotide function. The ways that an effective agent can act to modulate the expression of a polynucleotide include, but are not limited to 1) modifying binding of a transcription factor to a transcription factor responsive element in the polynucleotide; 2) modifying the interaction between two transcription factors necessary for expression of the polynucleotide; 3) altering the ability of a transcription factor necessary for expression of the polynucleotide to enter the nucleus; 4) inhibiting the activation of a transcription factor involved in transcription of the polynucleotide; 5) modifying a cell-surface receptor which normally interacts with a ligand and whose binding of the ligand results in expression of the polynucleotide; 6) inhibiting the inactivation of a component of the signal transduction cascade that leads to expression of the polynucleotide; and 7) enhancing the activation of a transcription factor involved in transcription of the polynucleotide.

A “function” of a polypeptide includes, but is not limited to, conformation, folding (or other physical characteristics), binding to other moieties (such as ligands), activity (or other functional characteristics), and/or other aspects of protein structure or functions. For example, an agent that acts on a polypeptide and affects its conformation, folding (or other physical characteristics), binding to other moieties (such as ligands), activity (or other functional characteristics), and/or other aspects of protein structure or functions is considered to have modulated polypeptide function. The ways that an effective agent can act to modulate the function of a polypeptide include, but are not limited to 1) changing the conformation, folding or other physical characteristics; 2) changing the binding strength to its natural ligand or changing the specificity of binding to ligands; and 3) altering the activity of the polypeptide.

Generally, the choice of agents to be screened is governed by several parameters, such as the particular polynucleotide or polypeptide target, its perceived function, its three-dimensional structure (if known or surmised), and other aspects of rational drug design. Techniques of combinatorial chemistry can also be used to generate numerous permutations of candidates. Those of skill in the art can devise and/or obtain suitable agents for testing.

The in vivo screening assays described herein may have several advantages over conventional drug screening assays: 1) if an agent must enter a cell to achieve a desired therapeutic effect, an in vivo assay can give an indication as to whether the agent can enter a cell; 2) an in vivo screening assay can identify agents that, in the state in which they are added to the assay system are ineffective to elicit at least one characteristic which is associated with modulation of polynucleotide or polypeptide function, but that are modified by cellular components once inside a cell in such a way that they become effective agents; 3) most importantly, an in vivo assay system allows identification of agents affecting any component of a pathway that ultimately results in characteristics that are associated with polynucleotide or polypeptide function.

In general, screening can be performed by adding an agent to a sample of appropriate cells which have been transfected with a polynucleotide identified using the methods of the present invention, and monitoring the effect, i.e., modulation of a function of the polynucleotide or the polypeptide encoded within the polynucleotide. The experiment n some embodiments includes a control sample which does not receive the candidate agent. The treated and untreated cells are then compared by any suitable phenotypic criteria, including but not limited to microscopic analysis, viability testing, ability to replicate, histological examination, the level of a particular RNA or polypeptide associated with the cells, the level of enzymatic activity expressed by the cells or cell lysates, the interactions of the cells when exposed to infectious agents, and the ability of the cells to interact with other cells or compounds. Differences between treated and untreated cells indicate effects attributable to the candidate agent. Optimally, the agent has a greater effect on experimental cells than on control cells. Appropriate host cells include, but are not limited to, eukaryotic cells, such as mammalian cells. The choice of cell will at least partially depend on the nature of the assay contemplated.

To test for agents that upregulate the expression of a polynucleotide, a suitable host cell transfected with a polynucleotide of interest, such that the polynucleotide is expressed (as used herein, expression includes transcription and/or translation) is contacted with an agent to be tested. An agent would be tested for its ability to result in increased expression of mRNA and/or polypeptide. Methods of making vectors and transfection are well known in the art. “Transfection” encompasses any method of introducing the exogenous sequence, including, for example, lipofection, transduction, infection or electroporation. The exogenous polynucleotide may be maintained as a non-integrated vector (such as a plasmid) or may be integrated into the host genome.

To identify agents that specifically activate transcription, transcription regulatory regions could be linked to a reporter gene and the construct added to an appropriate host cell. As used herein, the term “reporter gene” means a gene that encodes a gene product that can be identified (i.e., a reporter protein). Reporter genes include, but are not limited to, alkaline phosphatase, chloramphenicol acetyltransferase, .beta.-galactosidase, luciferase and green fluorescence protein (GFP). Identification methods for the products of reporter genes include, but are not limited to, enzymatic assays and fluorimetric assays. Reporter genes and assays to detect their products are well known in the art and are described, for example in Ausubel et al. (1987) and periodic updates. Reporter genes, reporter gene assays, and reagent kits are also readily available from commercial sources. Examples of appropriate cells include, but are not limited to, fungal, yeast, mammalian, and other eukaryotic cells. A practitioner of ordinary skill will be well acquainted with techniques for transfecting eukaryotic cells, including the preparation of a suitable vector, such as a viral vector; conveying the vector into the cell, such as by electroporation; and selecting cells that have been transformed, such as by using a reporter or drug sensitivity element. The effect of an agent on transcription from the regulatory region in these constructs would be assessed through the activity of the reporter gene product.

Besides the increase in expression under conditions in which it is normally repressed mentioned above, expression could be decreased when it would normally be expressed. An agent could accomplish this through a decrease in transcription rate and the reporter gene system described above would be a means to assay for this. The host cells to assess such agents would need to be permissive for expression.

Cells transcribing mRNA (from the polynucleotide of interest) could be used to identify agents that specifically modulate the half-life of mRNA and/or the translation of mRNA. Such cells would also be used to assess the effect of an agent on the processing and/or post-translational modification of the polypeptide. An agent could modulate the amount of polypeptide in a cell by modifying the turn-over (i.e., increase or decrease the half-life) of the polypeptide. The specificity of the agent with regard to the mRNA and polypeptide would be determined by examining the products in the absence of the agent and by examining the products of unrelated mRNAs and polypeptides. Methods to examine mRNA half-life, protein processing, and protein turn-over are well know to those skilled in the art.

In vivo screening methods could also be useful in the identification of agents that modulate polypeptide function through the interaction with the polypeptide directly. Such agents could block normal polypeptide-ligand interactions, if any, or could enhance or stabilize such interactions. Such agents could also alter a conformation of the polypeptide. The effect of the agent could be determined using immunoprecipitation reactions. Appropriate antibodies would be used to precipitate the polypeptide and any protein tightly associated with it. By comparing the polypeptides immunoprecipitated from treated cells and from untreated cells, an agent could be identified that would augment or inhibit polypeptide-ligand interactions, if any. Polypeptide-ligand interactions could also be assessed using cross-linling reagents that convert a close, but noncovalent interaction between polypeptides into a covalent interaction. Techniques to examine protein—protein interactions are well known to those skilled in the art. Techniques to assess protein conformation are also well known to those skilled in the art.

It is also understood that screening methods can involve in vitro methods, such as cell-free transcription or translation systems. In those systems, transcription or translation is allowed to occur, and an agent is tested for its ability to modulate function. For an assay that determines whether an agent modulates the translation of mRNA or a polynucleotide, an in vitro transcription/translation system may be used. These systems are available commercially and provide an in vitro means to produce mRNA corresponding to a polynucleotide sequence of interest After mRNA is made, it can be translated in vitro and the translation products compared. Comparison of translation products between an in vitro expression system that does not contain any agent (negative control) with an in vitro expression system that does contain an agent indicates whether the agent is affecting translation. Comparison of translation products between control and test polynucleotides indicates whether the agent, if acting on this level, is selectively affecting translation (as opposed to affecting translation in a general, non-selective or non-specific fashion). The modulation of polypeptide function can be accomplished in many ways including, but not limited to, the in vivo and in vitro assays listed above as well as in in vitro assays using protein preparations. Polypeptides can be extracted and/or purified from natural or recombinant sources to create protein preparations. An agent can be added to a sample of a protein preparation and the effect monitored; that is whether and how the agent acts on a polypeptide and affects its conformation, folding (or other physical characteristics), binding to other moieties (such as ligands), activity (or other functional characteristics), and/or other aspects of protein structure or functions is considered to have modulated polypeptide function.

In an example for an assay for an agent that binds to a polypeptide encoded by a polynucleotide identified by the methods described herein, a polypeptide is first recombinantly expressed in a prokaryotic or eukaryotic expression system as a native or as a fusion protein in which a polypeptide (encoded by a polynucleotide identified as described above) is conjugated with a well-characterized epitope or protein. Recombinant polypeptide is then purified by, for instance, immunoprecipitation using appropriate antibodies or anti-epitope antibodies or by binding to immobilized ligand of the conjugate. An affinity column made of polypeptide or fusion protein is then used to screen a mixture of compounds which have been appropriately labeled. Suitable labels include, but are not limited to fluorochromes, radioisotopes, enzymes and chemiluminescent compounds. The unbound and bound compounds can be separated by washes using various conditions (e.g. high salt, detergent) that are routinely employed by those skilled in the art. Non-specific binding to the affinity column can be minimized by pre-clearing the compound mixture using an affinity column containing merely the conjugate or the epitope. Similar methods can be used for screening for an agent(s) that competes for binding to polypeptides. In addition to affinity chromatography, there are other techniques such as measuring the change of melting temperature or the fluorescence anisotropy of a protein which will change upon binding another molecule. For example, a BIAcore assay using a sensor chip (supplied by Pharmacia Biosensor, Stitt et al. (1995) Cell 80: 661-670) that is covalently coupled to polypeptide may be performed to determine the binding activity of different agents.

It is also understood that the in vitro screening methods of this invention include structural, or rational, drug design, in which the amino acid sequence, three-dimensional atomic structure or other property (or properties) of a polypeptide provides a basis for designing an agent which is expected to bind to a polypeptide. Generally, the design and/or choice of agents in this context is governed by several parameters, such as side-by-side comparison of the structures of a prokaryote's and homologous closely related prokaryote's polypeptides, the perceived function of the polypeptide target, its three-dimensional structure (if known or surmised), and other aspects of rational drug design. Techniques of combinatorial chemistry can also be used to generate numerous permutations of candidate agents.

Also contemplated in screening methods of the invention are transgenic animal and plant systems, which are known in the art.

The screening methods described above represent primary screens, designed to detect any agent that may exhibit activity that modulates the function of a polynucleotide or polypeptide. The skilled artisan will recognize that secondary tests will likely be necessary in order to evaluate an agent further. For example, a secondary screen may comprise testing the agent(s) in an infectivity assay using mice and other animal models (such as rat), which are known in the art. In addition, a cytotoxicity assay would be performed as a further corroboration that an agent which tested positive in a primary screen would be suitable for use in living organisms. Any assay for cytotoxicity would be suitable for this purpose, including, for example the MTT assay (Promega).

The invention also includes agents identified by the screening methods described herein.

The following examples are provided to further assist those of ordinary skill in the art. Such examples are intended to be illustrative and therefore should not be regarded as limiting the invention. A number of exemplary modifications and variations are described in this application and others will become apparent to those of skill in this art. Such variations are considered to fall within the scope of the invention as described and claimed herein.

### EXAMPLES

### Example 1

### Obtaining Genomic Sequence Data for Bacillus anthracis and Bacillus cereus

Genomic sequence data from B. anthracis and B. cereus were downloaded from public databases maintained by the National Center for Biotechnology Information (NCBI), which maintains a website.

### Example 2

### Molecular Evolution Analysis

Ka/Ks values were calculated for homologous genes from B. anthracis and B. cereus using software, which aligns homologous sequences and then applies the Li algorithm to calculate the Ka/Ks values.

Seven potential candidate genes appear to have been positively selected in B. anthracis.

TABLE 1
Positions of positively selected genes in GenBank Accession #
AE016877 (Bacillus anthracis ATCC 14579 complete genome)

| | GenBank | | |
|---|---|---|---|
| Gene | Accession | Chromosomal | |
| number | Number | location | SEQ ID NO: |
| 1 | AE017030 | 135726-136886 | 1 |
| 2 | AE017034 | 144733-142403 | 7 |
| 3 | AE017035 | 26293-25940 | 13 |
| 4 | AE017028 | 260105-260719 | 19 |
| 5 | AE017039 | 45640-45386 | 25 |
| 6 | AE017029 | 55159-55431 | 31 |

### Example 3

### Analysis of Proteins Encoded by Positively Selected Genes

Significantly, some of the genes that were identified as positively selected may be relevant to recent research on anthrax virulence. At least two of the B. anthracis genes that appear to have been strongly positively selected (relative to their B. cereus homologs) encode putative proteases that could contribute to anthrax lethality. One of these is a bacterial metallopeptidase; homologs have been identified in a number of pathogenic bacteria. The second is involved in pathways that lead to production of bacterial toxins. Again, homologs are known from a number of pathogenic bacteria. Another candidate bears homology to a human protein involved in the coagulation cascade. Two of the candidate genes are unknown: no homologs have been reported.

### Example 4

### Validation

Genes identified are validated in suitable in vitro and/or in vivo models. One validation method is to knock out the six genes described above in B. anthracis. All of the six genes are knocked out, or, each individual gene or combinations of genes could be knocked out to assess their impact individually and in combination. Directed gene deletions can be accomplished using methods known to those skilled in the art, such as those reported in Cendrowski, S. et al. Molecular Microbiology January 2004 51 (2):407. Knock out B. anthracis mutants can be evaluated for virulence in an animal model, such as the Ames BALB/c mouse. Additionally, the six genes can be validated by substituting the B. cereus homolog of each of the six genes described in Example 3 into B. anthracis and virulence assessed in an animal model, such as the Ames BALB/c mouse. Gene substitution could be accomplished by homologous recombination. The genes could be assessed all together and individually, and, depending on experimental outcome, in different combinations.

### Example 5

### Screening for Agents

Screening will conducted to find compounds to combat the virulence that results from selected proteins, to be used as therapeutics. Knowledge of key virulence genes and their protein products will facilitate development of diagnostics for the rapid identification of B. anthracis. In addition, proteins identified will be used in the preparation vaccines.

Proteins encoded by the virulence genes identified above will be used in assays to screen for agents that bind to the proteins. Agents identified will be assessed in secondary assays using animal models such as the Ames BALB/c mouse. Agents that bind to the proteins and protect B. anthracis-infected mice from pathogenicity due to B. anthracis will be chosen for further development as potential human therapeutics.

### Example 6

### Diagnostics

B. anthracis virulence genes and will be used to design DNA probes for the rapid identification of B. anthracis, differentiated from closely related B. cereus and/or B. thuringensis in environmental samples or in medical samples. Similarly, proteins encoded by the genes will be used to identify polynucleotide or polypeptide binding agents for use in protein binding assays.

### Example 7

### Vaccines

The proteins encoded by the virulence genes identified above could be used to develop toxoid vaccines.

1. A method for identifying a polynucleotide sequence encoding a polypeptide associated with a virulence trait, comprising the steps of:
a) comparing polypeptide-coding polynucleotide sequences in a first prokaryote to polypeptide-coding polynucleotide sequences of a homologous genes of a second prokaryote, wherein said second prokaryote is less pathogenic relative to the first prokaryote; and
b) selecting a polynucleotide sequence in the first prokaryote that contains a nucleotide change as compared to the corresponding sequence of the second prokaryote, wherein said change is evolutionarily significant;
whereby a prokaryotic polynucleotide sequence encoding a polypeptide associated with a virulence trait is identified.

2. The method of claim 1, wherein the prokaryote is a member of the Bacillus genus.

3. The method of claim 2, wherein the prokaryote is B. anthracis.

4. The method of claim 1, wherein the nucleotide change is a non-synonymous substitution.

5. The method of claim 1, wherein the evolutionary significance of the nucleotide change is determined according to the non-synonymous substitution rate (Ka) of the nucleotide sequence.

6. The method of claim 5, wherein the evolutionary significance of the nucleotide change is determined by the ratio of the non-synonymous substitution rate (Ka) to the synonymous rate (Ks) of the nucleotide sequence.

7. The method of claim 6, wherein the Ka/Ks ratio is at least about 1.00.

8. The method of claim 6, wherein the Ka/Ks ratio is at least about 1.25.

9. The method of claim 6, wherein the Ka/Ks ratio is at least about 1.50.

10. The method of claim 6, wherein the Ka/Ks ratio is at least about 2.00.

11. A method of identifying an agent which may modulate virulence, said method comprising contacting at least one agent to be tested with a cell that has been transfected with a polynucleotide sequence identified in claim 1, wherein an agent is identified by its ability to modulate function of the polynucleotide sequence.

12. The method of claim 11, wherein the polynucleotide is selected from the group consisting of
a) a polynucleotide selected from the group consisting of SEQ ID NO:1, SEQ ID NO:7, SEQ ID. NO:13, SEQ ID NO:19, SEQ ID NO:25, and SEQ ID NO:31; and
b) a polynucleotide having at least 85% homology to a polynucleotide of a), and which confers substantially the same virulence as the polynucleotide of a).

13. A method of identifying an agent which may modulate virulence, said method comprising contacting at least one agent to be tested with a polypeptide encoded within a polynucleotide sequence identified in claim 1, or a composition comprising said polypeptide, wherein an agent is identified by its ability to modulate function of the polypeptide sequence.

14. The method of claim 13, wherein the polypeptide is selected from the group consisting of
a) a polypeptide encoded by a polynucleotide selected from the group consisting of SEQ ID NO:1, SEQ ID NO:7, SEQ ID. NO:13, SEQ ID NO:19, SEQ ID NO:25, and SEQ ID NO:31;
b) a polypeptide having at least 85% homology to a polypeptide of a), and which confers substantially the same virulence as the polypeptide of a); and
c) a polypeptide selected from the group consisting of SEQ ID NO:3, SEQ ID NO:9, SEQ ID. NO:15, SEQ ID NO:21, SEQ ID NO:27, and SEQ ID NO:33.

15. A method for correlating an evolutionarily significant prokaryotic nucleotide change to a virulence trait, comprising:
analyzing a functional effect, if any, of a polynucleotide sequence identified in claim 1 in a suitable model system, wherein presence of a functional effect indicates a correlation between the evolutionarily significant nucleotide change and the virulence trait.

16. The method of claim 15, wherein the polynucleotide is selected from the group consisting of
a) a polynucleotide selected from the group consisting of SEQ ID NO:1, SEQ ID NO:7, SEQ ID. NO:13, SEQ ID NO:19, SEQ ID NO:25, and SEQ ID NO:31; and
b) a polynucleotide having at least 85% homology to a polynucleotide of a), and which confers substantially the same virulence as the polynucleotide of a).

17. A method for correlating an evolutionarily significant prokaryotic nucleotide change to a virulence trait, comprising:
analyzing a functional effect, if any, of a polypeptide encoded in a polynucleotide sequence identified in claim 1 in a suitable model system, wherein presence of a functional effect indicates a correlation between the evolutionarily significant nucleotide change and the virulence trait.

18. The method of claim 17, wherein the polypeptide is selected from the group consisting of
a) a polypeptide encoded by a polynucleotide selected from the group consisting of SEQ ID NO:1, SEQ ID NO:7, SEQ ID. NO:13, SEQ ID NO:19, SEQ ID NO:25, and SEQ ID NO:31;
b) a polypeptide having at least 85% homology to a polypeptide of a), and which confers substantially the same virulence as the polypeptide of a); and
c) a polypeptide selected from the group consisting of SEQ ID NO:3, SEQ ID NO:9, SEQ ID. NO:15, SEQ ID NO:21, SEQ ID NO:27, and SEQ ID NO:33.
